(19) United States
(12) Patent Application Publication
(10) Pub. No.: US 2010/0105993 A1
(43) Pub. Date: Apr. 29, 2010

(54) METHODS AND APPARATUS FOR NONINVASIVE ISCHEMIC CONDITIONING

(75) Inventors: Morteza Naghavi, Houston, TX (US); Albert Andrew Yen, Houston, TX (US); Haider Hassan, Houston, TX (US); David Panthagani, Houston, TX (US)

Correspondence Address: WONG, CABELLO, LUTSCH, RUTHERFORD & BRUCCULERI,;L.L.P., 20333 SH 249 6th Floor, HOUSTON, TX, 77070, US

(73) Assignee: IC THERAPEUTICS, INC., Houston, TX (US)

(21) Appl. No.: 12/601,509

(22) Filed: Nov. 23, 2009

Related U.S. Application Data
(63) Continuation-in-part of application No. PCT/US2008/064767, filed on May 23, 2008.
Application No. PCT/US2008/064767 is a continuation-in-part of application No. 12/323,392, filed on Nov. 25, 2008.
Application No. 12/323,392 is a continuation-in-part of application No. 12/511,976, filed on Jul. 29, 2009.
(60) Provisional application No. 61/029,147, filed on Feb. 15, 2008.
(60) Provisional application No. 61/025,715, filed on Feb. 1, 2008.
(60) Provisional application No. 60/969,863, filed on Sep. 4, 2007.
(60) Provisional application No. 60/939,821, filed on May 23, 2007.
(60) Provisional application No. 60/989,946, filed on Nov. 25, 2007.
(60) Provisional application No. 61/188,043, filed on Aug. 6, 2008.

(51) Int. Cl. A61B 5/00

(52) U.S. Cl. 600/301

(57) ABSTRACT

A method for ischemic conditioning treatment in a patient is provided. Transient ischemia is caused by interrupting blood flow to a tissue and a response to the ischemic conditioning treatments is monitored and the ischemia and response thereto is adjusted and controlled based on the monitoring results.

### CROSS REFERENCE TO RELATED APPLICATIONS

This application claims priority based on U.S. Provisional Application No. 60/939,821 filed May 23, 2007, U.S. Provisional Application No. 60/969,863 filed Sep. 4, 2007, U.S. Provisional Application No. 61/025,715 filed Feb. 1, 2008, and U.S. Provisional Application No. 61/029,147 filed Feb. 15, 2008, the disclosures of which are incorporated herein by reference in their entireties.

### BACKGROUND

The disclosures herein relate generally to noninvasive ischemic conditioning and more particularly to methods and apparatus to prevent, reduce, and/or treat complications of one or more medical conditions.

Brief periods of ischemia (a local shortage of oxygen-carrying blood supply) in biological tissue are known in some systems to render that tissue more resistant to subsequent ischemic insults. Further, for an organ or tissue already undergoing total or subtotal ischemia, blood flow conditions can be modified during the onset of resumed blood flow to significantly reduce reperfusion injury. Since this method begins at the onset of resuming blood flow after ischemia, it is known as postconditioning.

Ischemic conditioning exerts tissue protection and appears to be a ubiquitous endogenous protective mechanism at the cellular level that has been observed in the heart of humans and other animal species tested. This protection has also been seen in organs such as the stomach, liver, kidney, gut, skeletal tissue, urinary bladder and brain. See D M Yellon and J M Downey, “Preconditioning the myocardium: from cellular physiology to clinical cardiology,” Physiol Rev 83 (2003) 1113-1151.

What are needed are apparatus and methods that adapt pre and post ischemic conditioning phenomena to novel applications in the prevention, reduction and treatment of disease conditions.

### SUMMARY

Provided herein are methods and apparatus for ischemic conditioning to reduce damage to tissues and/or improve response to therapies. In one embodiment, ischemic conditioning is effected by transiently and repeatedly administering transient ischemia to at least one vascular area of a patient or part thereof. In an embodiment, protective and/or therapeutic effects of ischemic conditioning can be enhanced by adjusting duration and frequency of ischemic conditioning protocols over a period of time. In an embodiment, effects of ischemic conditioning can be enhanced by administering multiple ischemic conditioning protocols over a period of time.

In an embodiment, an ischemic conditioning protocol can be specifically adapted to provide both early and delayed protective effects. In an embodiment, an ischemic conditioning protocol is adapted for occlusion of capillaries based on external pressure. In an embodiment, an ischemic conditioning protocol is implemented by a programmed device that is capable of tissue ischemia monitoring. In an embodiment, monitoring of oxygenation and metabolic markers of tissue ischemia is provided simultaneously with ischemic conditioning. In an embodiment, an ischemic conditioning protocol is adjusted based on monitoring of desired tissue markers, including but not limited to tissue markers of ischemia and metabolism. Alternatively, the invention is provided with monitoring of pulse or blood flow where partial occlusion is utilized.

The protective and therapeutic effects conferred by ischemic conditioning can be systemic or local to the ischemic tissue. In an embodiment, the ischemic conditioning can is administered to the location of an anticipated tissue injury. In another embodiment, the ischemic conditioning is administered after an intervention, such as chemotherapy, surgery, or occurrence of a wound. Ischemic conditioning can be administered to a tissue or organ that is a donor or recipient for a transplant, implant, or graft.

In an embodiment, the ischemic conditioning is remotely administered. In an embodiment, the ischemic conditioning is administered to a location on one or more limbs prior to chemotherapy. In an embodiment, the ischemic conditioning is a supplemental therapy to conventional therapies, such as using heat, photo thermal energy, hypoxia, pressure, vibration, or treatment drugs including anesthetics, anti-inflammatories, or compounds that increase the bioavailability of nitric oxide (NO), and combinations thereof.

In an embodiment, a device for ischemic conditioning is provided. In one embodiment the device has one or more occluding members in addition to controlling members. The controlling member may be programmable and the device may further include data storage members. A sensor for monitoring of tissue markers may be additionally provided. The occluding member may be adapted to at least partially occlude an internal vascular lumen to reduce or occlude flow to at least one peripheral tissue of the patient. In an embodiment, external skin pressure is provided to induce ischemia only at the skin and/or subdermal levels. The programmable controlling member may be adapted to control the frequency and duration of ischemia in a tissue according to an ischemic conditioning protocol. In an embodiment, the programmable controlling member is programmed by a separate device. The data storage member, such as a computer, may be adapted to store the protocol and/or monitoring results. An optional display may be provided to show the ischemic conditioning protocol, stored data, results of the ischemic conditioning, and/or other relevant data. The devices described herein may adapted for home or clinical use. For example, a device for home use may simply utilize external cuff occlusions around an extremity, blood pressure measurement, and/or pulse monitoring.

In one embodiment, methods and apparatus are provided for reduction of peripheral nerve damage complications induced by a pharmacologic or surgical intervention in a patient comprising by administering at least one vascular or neurovascular conditioning treatment to at least one distal extremity of the patient prior to the intervention. The treatment includes a least one vascular conditioning treatment by one or more of: induced ischemia or hypoxia, application of heat, photo thermal energy, vibration, or pressure, and administration of a drug selected from the group consisting of: vasodilating agents, anti-oxidants, anti-inflammatories, and compounds that increase the bioavailability of nitric oxide (NO) including nitric oxide donors, precursors and agonists. The vascular conditioning treatment in one embodiment is transiently and repeatedly applied according to a schedule that is implemented prior to initiating the intervention in the patient.

In one embodiment wherein the vascular conditioning treatment includes induced ischemia the induced ischemia is sufficient to induce reactive hyperemia in the distal extremity including both hands, both feet, or both hands and feet, and/or portions thereof. The method may be complemented by instructing a schedule of hand exercises to the patient.

In one embodiment employing induced ischemia as a preconditioning treatment, the induced ischemia is transiently and repeatedly induced in at least one limb or portion thereof of a patient according to a schedule of vascular occlusions prior to initiating the intervention in the patient. Alternatively or in addition to other preconditioning treatments, in one embodiment heat sufficient to induce vasodilatation is applied to at least one distal extremity of the patient. The heat may be generated by electric heating, ultrasound, microwave (MW), photo thermal energy, infrared (IR), radio frequency (RF) energy or heat derived from chemical reactions such as oxidation.

In other embodiments, pharmacotherapy is used as the preconditioning treatment or is initiated in conjunction with other treatment such as ischemic or heat treatment. The preconditioning pharmacotherapy is effected by administration with one or more of the group of vasodilators, anti-oxidants, anti-inflammatory and anesthetic compounds and nitric oxide (NO) donors, precursors and agonists. In one embodiment, the pharmacotherapy is administered systemically. In other embodiments, the pharmacotherapy is administered locally through the skin, such as by iontophoresis, wherein the driving electrical field is delivered through a glove and/or sock like garment.

In one embodiment an apparatus is provided for transiently inducing ischemia in a peripheral vascular area of a patient, the apparatus including a plurality of releasable compressing elements, each adapted to reduce or occlude flow of blood to at least a portion of a distal extremity of the patient. The releasable compressing elements may comprise bands dimensioned to be tightened around at least extremity such as an arm and/or leg to occlude flow of blood to at least a portion of the hands and/or feet. Alternatively, the releasable compressing elements may comprise inflatable compression mittens, socks and/or gloves. In one embodiment of the invention, apparatus for transiently and repeatedly inducing ischemia in a peripheral vascular area of a patient includes use of a plurality of releasable bands, each adapted to occlude blood supply to at least a hand or foot of the patient when tightened or inflated. As used here, the term band includes cuffs such as inflatable blood pressure type cuffs. The apparatus may be manual in operation or may be automated such as with a control device for controlling compressing and release elements in accordance with a schedule. In one embodiment the apparatus further includes a pump in operable communication with the releasable compressing elements, wherein the action of the pump results in tightening or inflation by filling of the releasable compressing elements. The apparatus may further include one or more heating mechanisms for intermittent heating at least one hand or foot.

In other embodiments of the invention, apparatus for transiently and repeatedly inducing heat in a peripheral vascular area of a patient is provided that includes use of a plurality of heating elements such that both hands and/or feet are transiently heated. The apparatus may be manual in operation or may be automated. In one embodiment the apparatus includes a programmable monitor for instructing heating in accordance with a schedule.

### BRIEF DESCRIPTION OF THE DRAWINGS

FIG. 1 depicts several locations for placement of noninvasive cuffs for inducing ischemic conditioning.

FIG. 2A depicts an embodiment of inflatable cuffs that can be curved when flat and closed by fasteners to be conical and/or adjustable. FIG. 2B depicts an embodiment of inflatable thigh cuffs that are secured to a molding.

FIG. 3A depicts an ambulatory embodiment enabling the patient to wear one or more noninvasive cuffs together with a controlling unit for scheduled inflation of the noninvasive cuff(s). FIG. 3B depicts two locations for placement of cuffs on arms. FIG. 3C depicts an embodiment of the placement of cuffs on two arms and two legs for ischemic conditioning.

FIG. 4A depicts a schematic of an example of early, or acute, and delayed SWOP therapeutic effects to be expected upon a single administration of ischemic conditioning. FIG. 4B depicts a schematic of an example of a “stacking” effect that is to be expected upon two or more administrations of ischemic conditioning.

FIG. 5A depicts an embodiment of a device that is implemented for home use of ischemic conditioning. FIG. 5B depicts another embodiment of a device that is implemented for home use of ischemic conditioning. FIG. 5C depicts an embodiment of a device that is implemented for home use which can also be calibrated or programmed by a separate device that is capable of tissue ischemia monitoring.

FIG. 6 depicts a system for ischemic conditioning.

FIG. 7 depicts an example of thresholds of ischemic effect on a tissue with which an ischemic conditioning protocol can be adjusted to prevent or reduce tissue injury.

FIG. 8A depicts cross sectional views of an embodiment of applying superficial pressure around an extremity. FIGS. 8B-D depict cross sectional views of embodiments of superficial pressure as applied against a body surface such as the skin.

FIG. 9A depicts placement of implementations that be adapted for treatments including inflation to drive blood from surface tissues and heating to induce vasodilation, both a prophylaxis. FIG. 9B depicts a glove implementation wherein each finger is isolated. FIGS. 9C and 9D depict cap implementations.

FIGS. 10-14 depict other embodiments for inflatable compression of the arm and hand for ischemic conditioning.

FIG. 15 depicts an embodiment of a pressured body suit that delivers external pressure to create ischemia at the skin and subdermal tissue levels.

FIGS. 16A-B depict embodiments of a mattress capable of preventing or reducing bedsores by ischemic conditioning.

FIGS. 17A-F show data indicating variations in tissue oxygenation between individuals.

FIG. 18A depicts an embodiment of an iontophoresis glove for drug conditioning in conjunction with ischemic conditioning. FIG. 18B depicts an embodiment of a toe cap that can be used for heating prophylaxis or iontophoresis drug conditioning in conjunction with ischemic conditioning. FIG. 18C depicts an embodiment of an iontophoresis drug delivery device dimensioned to fit over a digit such as a finger while FIG. 18D depicts an embodiment dimensioned for use over several digits.

### DETAILED DESCRIPTION

Without limiting the scope of the invention, the invention is described in connection with ischemic preconditioning and postconditioning for prevention or reduction of tissue injury complications, including those that result from known interventions. Ischemic preconditioning is a remarkable medical phenomenon. Eliciting brief periods of ischemia (a local shortage of oxygen-carrying blood supply) in biological tissue will render that tissue more resistant to subsequent ischemic insults. This method is known as preconditioning. Further, for an organ or tissue already undergoing total or subtotal ischemia, blood flow conditions can be modified during the onset of resumed blood flow to significantly reduce reperfusion injury. Since this method begins at the onset of resuming blood flow after ischemia, it is known as postconditioning.

The present inventors have adapted the experimental phenomena of ischemic conditioning to useful preventative and therapeutic measures for a myriad of novel indications. In certain embodiments, the process is monitored and controlled as well as individualized to the unique physiology of individual patients. The controlled induced ischemia disclosed and implemented herein provides conditioning to increase effects of therapies and decrease the incidence and extent of tissue injury by several mechanisms, e.g. increased scavenging of free radicals induced by trauma and reduction in inflammation. In other embodiments, the administration of controlled induced ischemia is adapted to increase functional capillary density in desired sites with an outcome of hastened wound healing. As used herein the term “ischemia” means lowering of baseline blood flow to a tissue. The term “hypoxia” means lowering of arterial PO2. Both ischemia and hypoxia in distal extremities can be induced by partial or complete occlusion of blood supply upstream of the extremity.

By “distal extremity” it is meant the hands and feet, including the digits of the hands and feet. By “regional or local” it is meant administration to a defined area of the body as contrasted with systemic administration. In an embodiment the occlusion is sufficient to induce reactive hyperemia in at least one limb or distal extremity thereof. “Reactive hyperemia” is a term that can be defined as an increase in blood flow to an area that occurs following a brief period of ischemia (e.g., arterial occlusion). One embodiment of the present invention employs controlled administrations of ischemia to condition tissues of target areas. By “target areas” it is meant areas known to exhibit injury expected to tissues during medical, surgical and other pharmacological interventions or non-pharmacological injuries. The term “ischemic conditioning” means inducing one or more episodes of ischemia that are controlled, including by monitoring of one or more biochemical markers in a target area.

As used herein the phrase “compounds that increase the bioavailability of nitric oxide (NO)” include NO precursors, NO donors and NO agonists. An example of a NO precursor is the essential amino acid substrate L-arginine from which NO is synthesized by the action of nitric oxide synthase (NOS). NO donors, which generate NO via NOS independent processes, include both fast and slow release compounds that typically release NO by either oxidation or reduction. Certain of the NO donor compounds such as nitroglycerin (an organic nitrate), which is enzymatically degraded to generate NO, have been utilized for over a century. Examples of NO donors (sometimes alternatively referred to in art as NO agonists) include the organic nitrates (e.g. glyceryl trinitrate, isosorbide dinitrate), sodium nitroprusside (SNP), syndnonimines (e.g. molsidomine, SIN-1), S-nitrosothiols (e.g. s-nitrosoglutathione), NONOates (e.g. Spermine-NONOate, DETA-NONOate), and hybrid donors such as the nitroaspirins and nicrorandil. Certain other compounds that are considered herein to fall within the definition of compounds that increase the bioavailability of NO are compounds, and metabolites thereof, that include nitric oxide chemical structures and are considered to be NO agonists such as for example minoxidil (3-hydroxy-2-imino-6-(1-piperidyl)pyrimidin-4-amine). Such compounds are considered herein to be NO agonists if their action is the same as NO, such as for example, in opening of membrane potassium channels.

Ischemic Preconditioning: The benefits of ischemic preconditioning have been observed in myocardial tissue of dogs that were pretreated by alternately manually clamping and unclamping coronary arteries to intermittently turn off the blood flow to the heart. Dogs who were treated with an optimal number of four cycles of five-minute coronary occlusion followed by five-minute reperfusion, exhibited 75% smaller infarct sizes resulting from a subsequent forty-minute coronary occlusion. Fewer than four cycles of coronary occlusion resulted in insufficient preconditioning in the dog model. Myocardial tolerance to injury also develops in response to treatment that does not include coronary occlusion (i.e., ischemia) but otherwise increases demand for oxygenated blood. In dogs, a treatment comprising of five five-minute periods of tachycardia alternating with five minutes of recovery has also been shown to reduce infarct sizes.

The myocardial resistance to infarct resulting from brief periods of ischemia has been described in other animal species including rabbit, rat and pig. Ischemic preconditioning has also been demonstrated in humans. A second coronary occlusion during the course of coronary angioplasty often results in less myocardial damage than the first. Naturally occurring ischemic preconditioning of the myocardium has been found in humans suffering from bouts of angina.

Ischemic preconditioning occurs not only in myocardial tissue but also occurs in non-cardiac tissue including kidney, brain, skeletal-muscle, lung, liver and skeletal tissue. Further, resistance to infarct exists even in virgin tissue following brief ischemia in spatially remote cardiac or non-cardiac tissue. Ischemic preconditioning also exhibits a temporal reach: an early phase develops immediately within minutes of the preconditioning ischemic injury and lasts for a few hours, and a late phase develops approximately twenty four hours later and can last for several days.

Postconditioning: Timely reperfusion to reduce the duration of ischemia is the definitive treatment to prevent cellular injury and necrosis in an ischemic organ or tissue. However, defined as reperfusion injury, additional damage can occur to an organ by the uncontrolled resumption of blood flow after an episode of prolonged ischemia. This damage is distinct from the injury resulting from the ischemia per se. One hallmark of reperfusion injury is that it may be attenuated by interventions initiated before or during the reperfusion. Reperfusion injury results from several complex and interdependent mechanisms that involve the production of reactive oxygen species, endothelial cell dysfunction, microvascular injury, alterations in intracellular Ca2+ handling, changes in myocardial metabolism, and activation of neutrophils, platelets, cytokines and the complement system. Deleterious consequences associated with reperfusion include a spectrum of reperfusion-associated pathologies that are collectively called reperfusion injury. Reperfusion injury can extend not only acutely, but also over several days following a medical or surgical intervention.

For example, even with successful treatment of occluded vessels, a significant risk of additional tissue injury after reperfusion may still occur. Typically, reperfusion after a short episode of myocardial ischemia is followed by the rapid restoration of cellular metabolism and function. However, if the ischemic episode has been of sufficient severity and/or duration to cause significant changes in the metabolism and the structural integrity of tissue, reperfusion may paradoxically result in a worsening of function, out of proportion to the amount of dysfunction expected simply as a result of the duration of blocked flow. Although the beneficial effects of early reperfusion of ischemic myocardium with thrombolytic therapy, PTCA, or CABG are now well established, an increasing body of evidence indicates that reperfusion also induces an additional injury to ischemic heart muscle, such as the extension of myocardial necrosis, i.e., extended infarct size and impaired contractile function and metabolism. Hearts undergoing reperfusion after transplantation also undergo similar reperfusion injury events. Similar mechanisms of injury are observed in all organs and tissues that are subjected to ischemia and reperfusion.

Thus, in general, all organs undergoing reperfusion are vulnerable to reperfusion injury. Postconditioning is a method of treatment for significantly reducing reperfusion injury to an organ or tissue already undergoing total or subtotal ischemia. Postconditioning involves a series of brief, iterative interruptions in arterial reperfusion applied at the immediate onset of reperfusion. The bursts of reflow and subsequent occlusive interruptions last for a matter of seconds, ranging from at least around 60 second intervals in larger animal models to 5-10 second intervals in smaller rodent models. Preliminary studies in humans used 1 minute intervals of reperfusion and subsequent interruptions in blood flow during catheter-based percutaneous coronary intervention (PCI).

The spatial and temporal characteristics of ischemic preconditioning and postconditioning may be a manifestation of complex interactions between various underlying phenomena. The numerous biochemical and cellular mechanisms underlying the phenomena of ischemic conditioning are still being researched and are not fully understood. These research efforts have been motivated at least in part by the hope of developing pharmaceutical drugs which would provide the infarct sparing effect of ischemic conditioning.

Ischemic Conditioning Protection at the Cellular Level: Ischemia has been shown to produce tolerance to damage from subsequent ischemic damage. Ischemia Preconditioning (IPC) was first described by Murry et al who found that protection was conferred to ischemic myocardium by preceding brief periods of sublethal ischemia separated by periods of reperfusion. (Murry C E, Jennings R B, Reimer K A. Circulation 74(5) (1986) 1124-36). As a consequence of four five-minute episodes of regional ischemia in the canine myocardium, a net effect of 75 percent reduction in infarct size compared to a control group.

The protective effects of conditioning may be mediated by signal transduction changes to tissues. The current paradigm suggests that nonlethal episodes of ischemia reduce infarct size. Ischemic conditioning has been found to lead to the release of certain substances, such as adenosine and bradykinin. These substances bind to their G-protein-coupled receptors and activate kinase signal transduction cascades. See Id. These kinases converge on the mitochondria, resulting in the opening of the ATP-dependent mitochondrial potassium channel. See Garlid K D et al. “Cardioprotective effect of diazoxide and its interaction with mitochondrial ATP-sensitive K⁺ channels. Possible mechanism of cardioprotection.” Circ Res 81 (1997) 1072-1082. Reactive oxygen species are then released. See Vanden Hoek T L et al., “Reactive oxygen species released from mitochondria during brief hypoxia induce preconditioning in cardiomyocytes.” J Biol Chem 273 (1998) 18092-18098. Thus additional protective signaling kinases can be activated, such as heat shock inducing protein kinase C.

Further, the signaling kinases mediate the transcription of protective distal mediators and effectors, such as inducible nitric oxide synthase, manganese superoxide dismutase, heat-stress proteins and cyclo-oxygenase 2, which manifest 24-72 hours after infarction to provide late protection. Suggested mechanisms of how these signaling transduction pathways mediate protection and ultimately reduce infarct size include maintenance of mitochondrial ATP generation, reduced mitochondrial calcium accumulation, reduced generation of oxidative stress, attenuated apoptotic signaling and inhibition of mitochondrial permeability transition-pore (mPTP) opening. See D M Yellon and J M Downey, “Preconditioning the myocardium: from cellular physiology to clinical cardiology,” Physiol Rev 83 (2003) 1113-1151; Yellon D M, Hausenloy D J, “Realizing the clinical potential of ischemic preconditioning and postconditioning,” Nat Clin Pract Cardiovasc Med. 2(11)(2005) 568-75. It is also possible that alternative protective mechanisms of ischemic conditioning might exist that are independent of signal transduction pathways, such as those mediated by antioxidant and anti-inflammatory mechanisms, and so on.

Even further, formation of vascular collaterals is also induced by ischemia and hypoxia of blood vessels. Vascular endothelial growth factor (VEGF) production can be induced in cells that are not receiving enough oxygen. When a cell is deficient in oxygen, it produces the transcription factor Hypoxia Inducible Factor (HIF). HIF stimulates the release of VEGF among other functions including modulation of erythropoeisis. Circulating VEGF then binds to VEGF receptors on endothelial cells and triggers a tyrosine kinase pathway leading to angiogenesis.

Ischemia has been shown to produce tolerance to reperfusion damage from subsequent ischemic damage. One physiologic reaction to local ischemia in normal individuals is reactive hyperemia to the previously ischemic tissue. Arterial occlusion results in lack of oxygen (hypoxia) as well as an increase in vasoactive metabolites (including adenosine and prostaglandins) in the tissues downstream from the occlusion. Reduction in oxygen tension in the vascular smooth muscle cells surrounding the arterioles causes relaxation and dilation of the arterioles and thereby decreases vascular resistance. When the occlusion is released, blood flow is normally elevated as a consequence of the reduced vascular resistance.

Perfusion of downstream tissues is further augmented by flow-mediated dilation (FMD) of larger conduit arteries, which acts to prolong the period of increased blood flow. As a consequence of the elevated blood flow induced by reactive hyperemia, downstream conduit vessels undergo luminal shear stress. Endothelial cells lining the arteries are sensitive to shear stress and the stress induces in opening of calcium-activated potassium channels and hyperpolarization of the endothelial cells with resulting calcium entry into the endothelial cells, which then activates endothelial nitric oxide synthase (eNOS). Consequent nitric oxide (NO) elaboration results in vasodilation. Endothelium-derived hyperpolarizing factor (EDHF), which is synthesized by cytochrome epoxygenases and acts through calcium-activated potassium channels, has also been implicated in flow-mediated dilation. Endothelium derived prostaglandins are also thought to be involved in flow-mediated dilation.

Ischemia Preconditioning (IPC) has been found to have remote and systemic protective effects in both human and animal models. Transient limb ischemia (3 cycles of ischemia induced by cuff inflation and deflation) on a contralateral arm provides protection against ischemia-reperfusion (inflation of a 12-cm-wide blood pressure cuff around the upper arm to a pressure of 200 mm Hg for 20 minutes) induced endothelial dysfunction in humans and reduces the extent of myocardial infarction in experimental animals (four cycles of 5 minutes occlusion followed by 5 minutes rest, immediately before occlusion of the left anterior descending (LAD) artery). (Kharbanda R K, et al. Circulation 106 (2002) 2881-2883.)

Recent evidence in a skeletal muscle model has suggested that IPC results in increased functional capillary density, prevention of ischemia/reperfusion induced increases in leukocyte rolling, adhesion, and migration, as well as upregulation of expression of nNOS, iNOS, and eNOS mRNA in ischemia reperfusion injured tissue. (Huang S S, Wei F C, Hung L M. “Ischemic preconditioning attenuates postischemic leukocyte-endothelial cell interactions: role of nitric oxide and protein kinase C” Circulation Journal 70 (8) (2006) 1070-5). Research has also shown that ischemic preconditioning can result in elevations of heat shock proteins, antioxidant enzymes, Mn-superoxide dismutase and glutathione peroxidase, all of which provide protection from free radical damage. (Chen Y S et al. “Protection ‘outside the box’ (skeletal remote preconditioning) in rat model is triggered by free radical pathway” J. Surg. Res. 126 (1) (2005) 92-101).

Although originally described as conferring protection against myocardial damage, preconditioned tissues have been shown to result in ischemia tolerance through reduced energy requirements, altered energy metabolism, better electrolyte homeostasis and genetic re-organization, as well as reperfusion tolerance due to less reactive oxygen species and activated neutrophils released, reduced apoptosis and better microcirculatory perfusion compared to non-preconditioned tissue. (Pasupathy S and Homer-Vanniasinkam S. “Ischaemic preconditioning protects against ischaemia/reperfusion injury: emerging concepts” Eur. J. Vasc. Endovasc. Surg. 29 (2) (2005) 106-15).

Noninvasive Ischemic Conditioning: In an embodiment of the invention, apparatus for inducing ischemia in a peripheral tissue includes use of a plurality of releasable occluding members such as noninvasive cuffs, each adapted to occlude blood supply to an extremity or portions thereof, such as the hands and feet and portions thereof, when tightened. The occlusion of blood flow can be partial or complete. As used here, the term noninvasive cuff includes cuffs such as inflatable blood pressure type cuffs. In an embodiment the releasable noninvasive cuffs can be integrated to apply pressure within compressing gloves for at least one finger, toe, hand or foot. The noninvasive cuffs can be manual in operation or can be automated. In an embodiment the noninvasive cuffs include a programmable monitor for instructing tightening of the noninvasive cuffs in accordance with a schedule. The schedule can instruct tightening the noninvasive cuffs such as in either a cyclical or sustained manner. In some embodiments, a pump is included that inflates the noninvasive cuffs and thereby occludes the blood supply according to the schedule and can further include one or more distal sensory mechanisms, drug infusion systems, heating mechanisms for heating of at least one hand or foot, and combinations thereof.

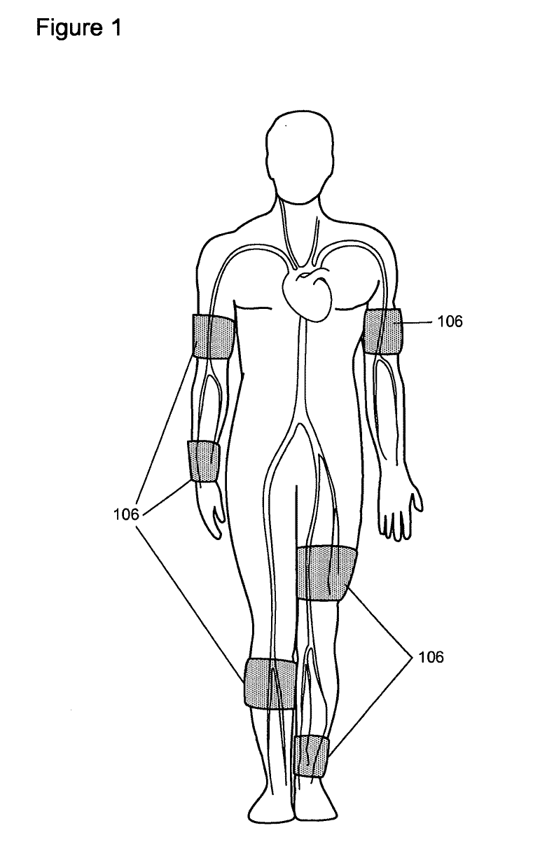

In an embodiment of the invention, ischemia is implemented by noninvasive cuffs or straps (106) that are secured around one or more of the limbs of the patient, as depicted in FIG. 1. As shown in FIG. 1, the noninvasive cuffs or straps can be placed over one or more locations for compression sufficient to occlude blood flow to the hands and or feet. For example, for occlusion of blood supply to the hands, compression can be applied to the upper arm over the brachial artery, the lower arm over the radial and ulnar arteries, or the wrist over the radial artery. For occlusion of blood supply to the feet, compression can be applied to the upper leg over the femoral and deep femoral arteries, or to the lower leg over the tibial arteries. In an embodiment, the noninvasive cuffs are inflatable and inflation results in sufficient pressure around the circumference of the limb to result in at least partial occlusion of the arterial blood supply to the limb.

The duration of ischemia varies by therapeutic targets, but is typically provided for a period from about 1 to about 20 minutes, preferably from about 2 to about 5 minutes, followed by release of the occlusion. Occlusion and release (reactive hyperemia) procedures with different occlusion times are implemented depending on individual tolerance and response to therapy as well as the planned treatment schedule such that a desired distal and or contralateral vascular/neuro/neurovascular function is obtained. Repeated cuff occlusion and release is tailored to improve vasoreactivity (increasing the vasodilative capacity) by improving nitric oxide bioavailability (by reducing destruction and/or increasing production). This effect can be seen in the same distal extremity as the cuff inflation but is also expected to have neurovascular mediated systemic vasodilation as well.

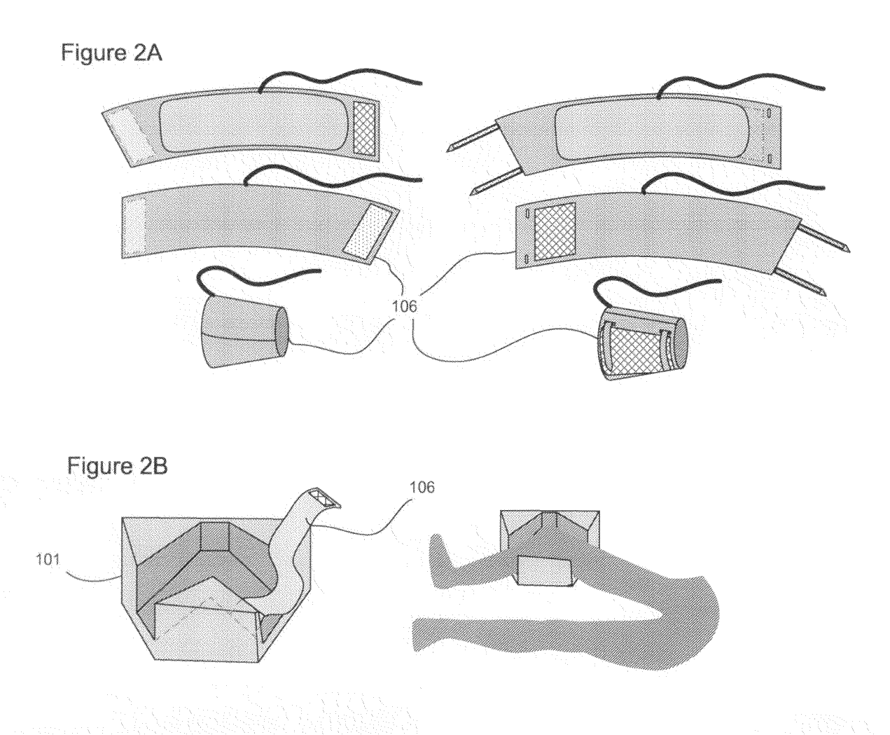

In one embodiment of the invention, a programmable cuff inflation and deflation device is employed to provide intermittent scheduled transient ischemia. The device can inflate one or more cuffs on one or more body parts at a time. The method induces reactive hyperemia and can mimic the effects of local exercise. The larger the area of ischemia the higher the hyperemia. In an embodiment, a cuff can be designed to reduce the discomfort of cuff occlusion by reducing the width of the cuff to alleviate pain from nerve pressure over a large area. However, the cuff can be designed to still be wide enough to avoid sharp pain and bruising as is often seen with a tourniquet. In an embodiment, a cuff can be placed on the upper part of the groin or upper arm where there is less muscle. In an embodiment, assuming the femoral arterial line runs towards the inside of the thigh, the side of the cuff contacting the inside of the thigh can contain a solid material slightly protruded to provide more pressure on the internal side. In an embodiment, a cuff can be adapted to provide such preferential pressure on any limb. In an embodiment, a cuff can be designed to reduce pain from cuff pressure through conical and/or adjustable shaping to better contour the limb. For example, FIG. 2A depicts an embodiment of inflatable cuffs (106) that can be curved when flat and closed by fasteners to be conical and/or adjustable. Further, in an embodiment, a cuff can be designed or adapted to inflate around a limb that is positioned in a manner to alleviate pain. For example, FIG. 2B depicts an embodiment of inflatable thigh cuffs that are secured to a molding (101). A patient can thus sit into this molding and be forced to rotate and bend the leg in a manner that will allow the cuff upon inflation to better contour the thigh. In an embodiment, any other suitable mechanism to bend a limb into occluding members can be adapted, e.g. cuffs that are connected in a sling or brace shape or cuffs adapted into a chair. In an embodiment, bending a limb during cuff occlusion can decrease the paresthesias and/or pain that results from nerve compression.

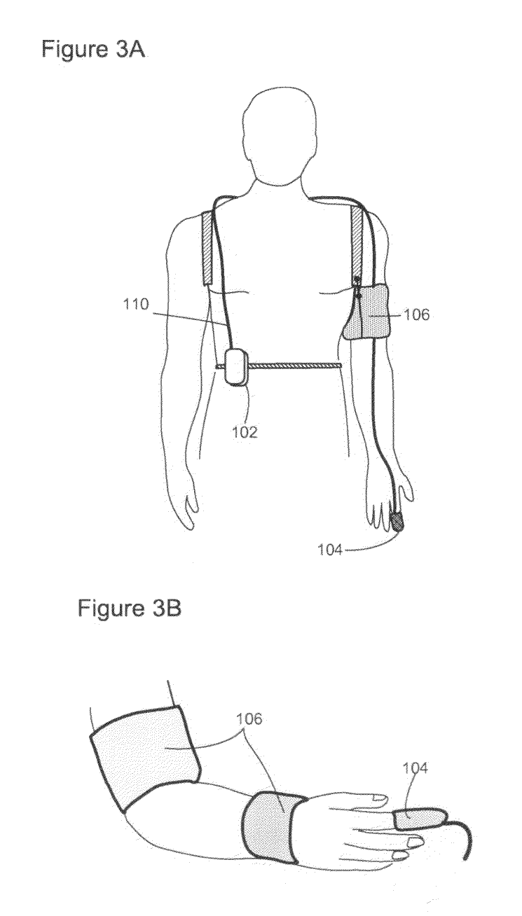

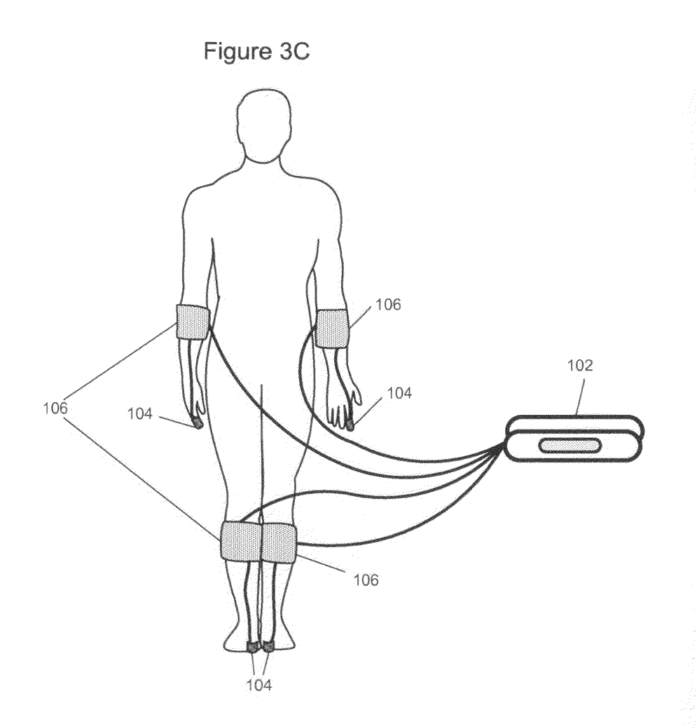

A form of the device is implemented for ambulatory use such as the embodiment depicted in part in FIG. 3A. In the embodiment depicted in FIG. 3A, one or more noninvasive cuffs (106) are in electrical connection via cable (110) with programmable controller (102) which can be worn anywhere on the body, such as for example on a waist as depicted. The inclusive noninvasive cuff can be tightened by an electric pump associated with the programmable monitor, which also can be adapted to record the pattern of occlusion. Alternatively, the noninvasive cuff(s) can be manually tightened at intervals. In an embodiment, the noninvasive cuff(s) are inflated in response to a signal given by a programmable monitor that instructs inflation and deflation of the inflatable noninvasive cuff(s). In another embodiment, the monitor instructs manual tightening and loosening of one or more straps in accordance with a programmed schedule. Further, to illustrate other cuff placements, FIG. 3B depicts an arm cuff embodiment in which reactive hyperemia is induced by cuffs (106) located on either the upper and/or lower arm. FIG. 3C depicts a non-ambulatory embodiment of the placement of a controller (102) and cuffs (106) on two arms and two legs for ischemic conditioning.

In an embodiment of the invention, as depicted in FIGS. 3A, 3B, and 3C, the hyperemic response induced by occlusive device (106) located either on an upper or lower limb is monitored, such as by a fingertip and/or toe monitor (104). Hyperemia can be monitored by any one of several methods that detect blood flow differences including inherent skin temperature, clearance of induced skin temperature, tonometry, Doppler ultrasound, laser Doppler flowmetry (such as by a laser Doppler perfusion imaging (LDPI) instrument), plethysmography, iontoporesis, capillometry, and/or changes in magnetic or electromagnetic properties of the tissue.

Inherent skin temperature means the unaltered temperature of the skin. This is in contrast to an induced skin temperature measurement which measures perfusion by clearance or wash-out of heat induced on the skin. Various methods of recording of inherent skin temperature on a finger tip or palm distal to an occlusive cuff are disclosed in Naghavi et al., U.S. application Ser. No. 11/563,676 and PCT/US2005/018437 (published as WO2005/118516). The combination of occlusive means and skin temperature monitoring for determination of vascular reactivity has been termed Digital Temperature Monitoring (DTM) by the present inventor.

Further, the duration of ischemia may not have a linear relationship with the effect. In an embodiment, after 30 minutes of complete occlusion, the hyperemia plateaus and the biochemical imprint changes. In an embodiment, desired therapeutic effects are more expected in the first 20 minutes after which the potential harm from the ischemia would have to be weighed against conditioning benefits. Similarly, in an embodiment, repeating cycles of noninvasive cuff occlusions and releases at a frequency faster than every 30 seconds or slower than every 36 hours can be ineffective.

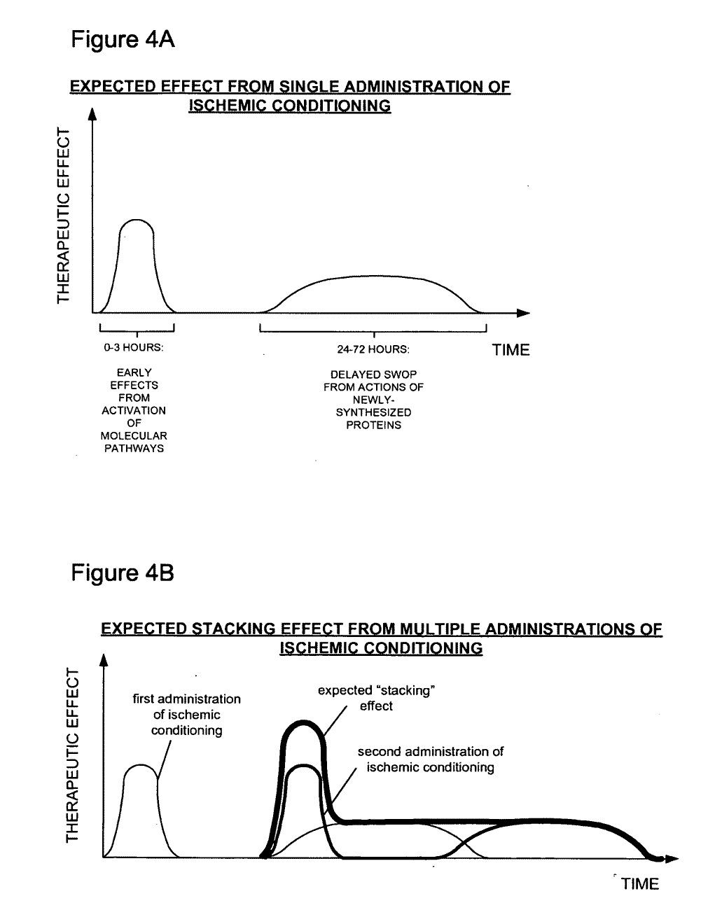

Alternatively, the duration and frequency of ischemia may have a relationship with the effect. Desired therapeutic effects within an early, or acute, window of 0-3 hours after conditioning and a delayed window, or second window of protection (SWOP), 24-72 hours after conditioning are expected due to the time difference of effectivity between activation of molecular pathways and newly-synthesized protein effectivity, respectively. FIG. 4A depicts a schematic of an example of early, or acute, and delayed SWOP therapeutic effects to be expected upon a single administration of ischemic conditioning. In an embodiment, multiple administrations of ischemic conditioning can be scheduled to provide an expected “stacking” effect where effectivity is expected to increase by combining a delayed SWOP effect of an earlier administration with an early effect of a later administration. For example, FIG. 4B depicts a schematic of an example of a “stacking” effect that is to be expected upon two administrations of ischemic conditioning. In an embodiment, scheduling several separate ischemic conditioning treatments apart from each other in a day (e.g. 3 times a day) can maximize therapeutic effect. In an embodiment, ischemic conditioning treatments are frequently conducted over extended periods of time (e.g. a day, month, or year) to enhance the therapeutic effect. In an embodiment, adjustments to ischemic conditioning regimens are administered based on assessments of specific interventions and/or treatment resistance over a period of time. For example, ischemic conditioning sessions of 4 cycles of 5 minute release and 5 minute occlusion of an extremity can be scheduled both 24 hours before and 1 hour before a surgery to achieve a combination of an early and delayed effect to improve perioperative outcomes. In one embodiment, at least one episode of transient ischemic conditioning is employed within 72 hours of a scheduled intervention. In other embodiments, a further episode is administered within the period of 72 hours following a scheduled intervention or other unscheduled traumatic event.

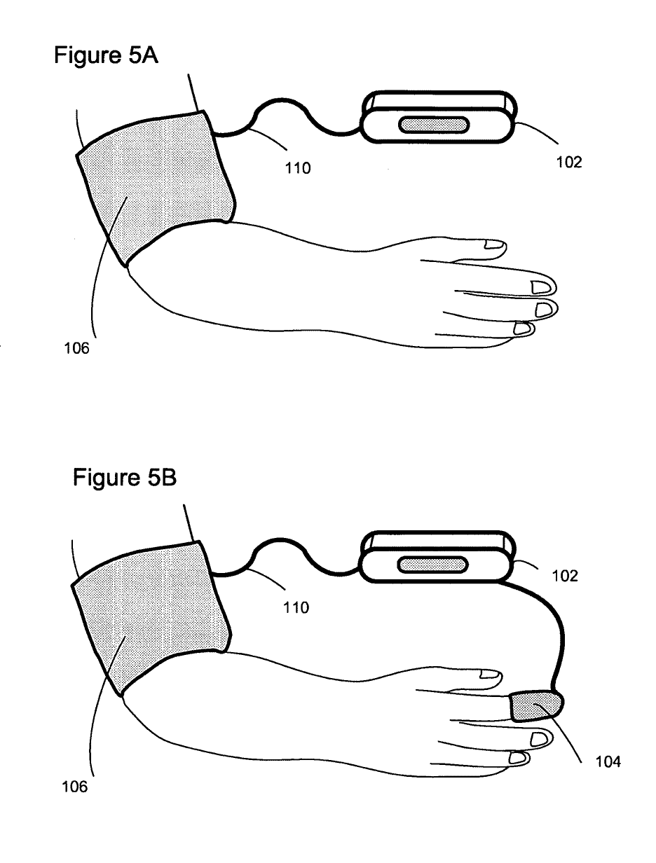

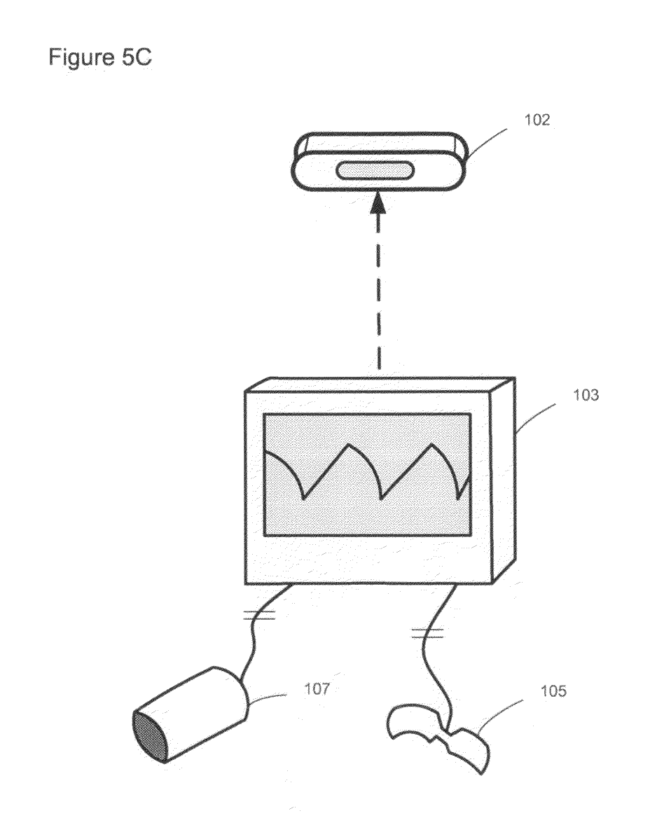

In one embodiment a form of the device is implemented for home use such as the embodiment depicted in part in FIG. 5A. In the embodiment depicted in FIG. 5A, one or more occlusive cuffs (106) are in electrical connection via cables (110) with programmable controller (102) which may be a desktop based device or worn anywhere on the body, such as for example on a waist band. FIG. 5B depicts use of a fingertip monitor (104) in conjunction with a blood flow occlusive device (106) located on an upper or lower extremity. In one embodiment, the method for monitoring the vascular or neurovascular response further includes simultaneously measuring and recording additional physiologic parameters including but not limited to pulse rate, blood pressure, galvanic response, sweating, core temperature, and/or skin temperature on a thoracic or truncal (abdominal) part.

Further, in an embodiment, a form of the device that is implemented for home use can be calibrated or programmed by a separate device that is capable of tissue ischemia monitoring, such as the embodiment depicted in part in FIG. 5C. In the embodiment depicted in FIG. 5C, one or more programmable monitors (102) are programmed or calibrated by a separate device (103) to adjust ischemic conditioning protocols, e.g. duration and frequency of occlusions, based on measurements of tissue ischemia. For example, as depicted, tissue ischemia is monitored using near infrared spectroscopy measurements via a NIRS sensor (105) to measure tissue oxygenation and metabolism, and a pulse oximeter (107) can monitor changes in blood flow, e.g. pulse. In an embodiment, the separate device can be adapted specifically for research and/or clinical settings.

Ischemic Conditioning Based on Monitoring of Tissue Markers: In accordance with the novel indication of the present invention, in one embodiment the individual patient's adaptive responses to induced ischemia or hypoxia are monitored to provide protection against tissue damage and to increase response to therapies. In an embodiment of the invention, duration and frequency of ischemia are adjusted based on monitoring of markers in a target tissue, including but not limited to metabolic, oxygenation, and/or biochemical markers. In an embodiment, supplemental episodes of heat, vibration, drugs, or combinations thereof, are provided based on monitoring of biochemical markers in the target tissue.

Several studies have indicated that there may be organ-specific biochemical thresholds for dysoxia, and yet heterogeneity of blood flow (or cellular metabolism) within an organ can also lead to different values at different locations within the same organ. For example, for a discussion of pH thresholds related to hepatic dysoxia. See, inter alia, Soller B R et al. “Application of fiberoptic sensors for the study of hepatic dysoxia in swine hemorrhagic shock.” Crit Care Med. 2001 July;29(7):1438-44. Further, overall tissue oxygen sufficiency can be confirmed by near-infrared measurement of cytochrome oxidase and the redox behavior of cytochrome oxidase during an operation is a good predictor of postoperative cerebral outcome. (Kakihana Y, et al., “Redox behavior of cytochrome oxidase and neurological prognosis in 66 patients who underwent thoracic aortic surgery.” Eur J Cardiothorac Surg. 2002 March;21(3):434-9.)

Accordingly, chronic, regular or periodic administration of ischemia can be optimized to suit the variable needs of the target area prior to an injurious intervention. For example, the individual patient may schedule a pattern of ischemia, such as for limited periods 5-10 times a day for a period preceding each intervention. In another embodiment, ischemia is administered to the future injury site for a period prior to injury. Depending on responses desired and obtained in the individual patient, the intensity and duration of ischemia can be tuned for optimal responses.

Further, in an embodiment, sensing and monitoring of markers can provide measurements to control ischemic preconditioning and postconditioning. In an embodiment, the target tissue has been at least partially damaged prior to inducing ischemia. In an embodiment, ischemia is controlled by postconditioning at the onset of reperfusion to reduce reperfusion injury. In an embodiment, ischemic preconditioning reduces damage to tissue due to a traumatic medical procedure such as surgery, angioplasty, chemotherapy, or radiation. In an embodiment, ischemia and heat can also be similarly adjusted to increase monitored effects of certain therapies, such as drugs and radiotherapy. For example, in an embodiment, neuropathy from chemotherapy and radiotherapy interventions can be reduced or prevented by providing ischemic preconditioning based on monitoring levels of oxygen in a target tissue.

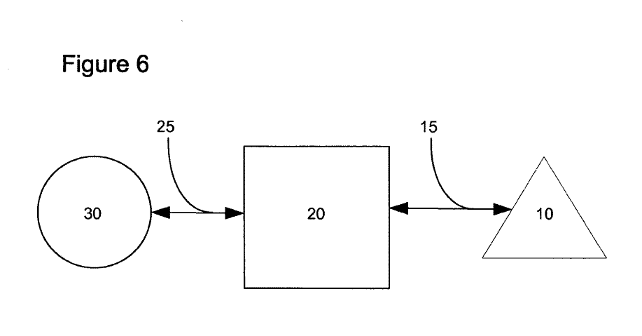

Ischemia can be controlled based on monitoring of biochemical markers by a system for ischemic conditioning. In an embodiment as depicted in FIG. 6, a system for ischemic conditioning can include an occluding device (10), a controlling device (20), a sensing device (30), and communication signals (15, 25) between the devices. The occluding device (10) induces ischemia through one or more episodes of occlusion of blood supply. The occluding device (10) is controllable by the controlling device (20) via a signal (15). The sensing device (30) is adapted to measure one or more biochemical markers in a target tissue and send information via a signal (25) to the controlling device (20). Accordingly, the controlling device (20) can control the one or more episodes of occlusion by the occluding device (10) based on monitoring of a signal (25) received from the sensing device (30).

Considering the occluding device in more detail, ischemia can be induced through one or more episodes of occlusion of blood supply by the occluding device. In an embodiment, the occluding device can be noninvasive. In an embodiment, the occluding device can induce occlusions at a duration and frequency suitable for the size of blood vessels and target tissue being conditioned. For example, in an embodiment, larger forearm arteries can be occluded at a longer duration and slower frequency than smaller blood vessels, such as those found in the fingers. In an embodiment, arterial occlusion is desirable in tissues with loose capillary walls as occlusion of the venous system in such tissues can result in unwanted leakage of plasma or blood into the tissue. However, in an another embodiment, to induce ischemia when arterial access for occlusion is unavailable, venous occlusion can be beneficial to prevent or reduce venous blood flow and in turn prevent or reduce arterial blood flow.

The duration and frequency of ischemia varies by therapeutic targets, but both duration and frequency of occlusions can be sustained for longer periods depending on the extent of occlusion. For example, within the same individual, the duration and frequency of ischemic conditioning can be adjusted to suit the faster metabolisms of tissues in the brain or heart as opposed to the slower metabolisms of other tissues, e.g. hair. Further, in an embodiment, the duration and frequency of ischemic conditioning can be adjusted to suit metabolic differences across individuals. Also, occlusion and release (reactive hyperemia) procedures with different durations and frequencies are implemented depending on individual tolerance and response to therapy. In an embodiment, duration and frequencies can vary upon a planned intervention schedule so that a desired distal and or contralateral vascular/neuro/neurovascular function is obtained. Occlusion and release is tailored to improve vasoreactivity (increasing the vasodilative capacity) by improving nitric oxide bioavailability (reducing destruction or increasing production). This effect can be seen in the same distal extremity as the occlusion but is also expected to have neurovascular mediated vasodilation of the contralateral extremity as well.

Considering the controlling device and sensing device in more detail, duration and frequency of ischemia and thermal conditioning can be adjusted by the controlling device based on monitoring of tissue markers of metabolic activity and/or therapeutic effects in the target tissue by the sensing device. For example, if levels of oxygen are monitored as dropping significantly into dysoxia and irreversible injury, the controlling device can alter ischemic episodes to decrease or stop until oxygen levels are monitored to be at a suitable range. Once reaching a desirable range, the ischemic episodes can resume under further monitoring. In an embodiment, a significant enough change in oxygen saturation levels to trigger a conditioning response can be at least 1%. In an embodiment, a significant enough change in oxygen saturation levels to trigger a conditioning response can vary depending on clinical conditions including areas of occlusion, areas of target tissue, duration and frequency of ischemia, and individual tolerance and response to therapy.

Similarly, if levels of other tissue markers of ischemia, including but not limited to lactate, pH, carbon dioxide, ATP, ADP, nitric oxide, peroxinitrate, electrolytes, free radicals, and combinations thereof, are determined to be changing significantly, the controlling device can adjust ischemic episodes until those levels are monitored to be at a suitable level again. Once reaching a desirable range, the ischemic episodes can resume under further monitoring. In an embodiment, a significant enough change in saturation levels of any marker to trigger a conditioning response can be at least 1%. In an embodiment, a significant enough change in saturation levels of markers to trigger a conditioning response can vary depending on clinical conditions including areas of occlusion, the particular target tissue, and duration and frequency of ischemia.

Further, if levels of other tissue markers of ischemic conditioning therapy, including but not limited to responses to chemotherapy, radiotherapy, neuropathy, hypertension, chronic conditions, operative outcome, and/or wound healing, are determined to be changing significantly, the controlling device can adjust ischemic episodes until those levels are monitored to be at a suitable level again. Once reaching a desirable range, the ischemic episodes can resume under further monitoring. For example, if tissue markers of chemotherapy induced neuropathy indicate an increase in tissue injury, the frequency of ischemic conditioning treatments can be decreased to prevent or reduce such injury. In an embodiment, measurement of tissue markers of response to ischemic conditioning treatments can include but are not limited to: adenosine, cytochrome oxidase, redox voltage, erythropoietin, bradykinin, opioids, ATP/ADP, and/or related receptors.

Monitoring can be continuous or intermittent, depending on the target tissues and the character of the intervention. For example, monitoring of tissues with slower inherent metabolic rate can be undertaken with more intermittent monitoring than those with high metabolic rates, such as cardiac tissue. Thus, in an embodiment, the desired frequency of monitoring of markers can depend on the extent of the induced ischemia and target tissue areas. In an embodiment, monitoring of tissue markers can provide data to satisfy thresholds of ischemia to adjust the ischemic conditioning protocol in order to prevent or minimize cell injury. For example, FIG. 7 depicts an example of thresholds of ischemic effect on a tissue with which an ischemic conditioning protocol can be adjusted to prevent or reduce tissue injury.

In an embodiment, biochemical markers in the target tissue include levels of lactate, pH, oxygen, carbon dioxide, ATP, ADP, nitric oxide, peroxinitrate, electrolytes, free radicals, and combinations thereof. In an embodiment, anaerobic conditions during ischemia can change levels of these biochemical markers of metabolic activity in the target tissue. For example, anaerobic respiration can cause lactate levels to increase, pH levels to decrease, oxygen levels to decrease, ATP levels to decrease, and ADP levels to increase. Other biochemical changes can also be measured in the target tissue, such as shifted levels of nitric oxide and peroxinitrate, electrolytes, and free radical redox states. Further, in an embodiment, the induced ischemia is modified and controlled until levels of the biochemical markers are measured to return to desirable ranges.

In an embodiment, biochemical marker measurement can also include thermal markers in the target tissue. Thermal markers can include levels of perfusion, carbon dioxide, external and inherent temperatures, and combinations thereof. Inherent skin temperature means the unaltered temperature of the skin. This is in contrast to an induced skin temperature measurement which measures perfusion by clearance or wash-out of heat induced on the skin. Various methods of recording of inherent skin temperature on a finger tip or palm distal to a noninvasive cuff are disclosed in Naghavi et al., U.S. application Ser. No. 11/563,676 and PCT/US2005/018437 (published as WO2005/118516). The combination of occlusive means and skin temperature monitoring has been termed Digital Temperature Monitoring (DTM) by the present inventor. In an embodiment, the method for monitoring the hyperemic response further includes simultaneously measuring and recording additional physiologic parameters including but not limited to pulse rate, blood pressure, galvanic response, sweating, core temperature, and/or skin temperature on a thoracic or truncal (abdominal) part.

In an embodiment, tissue markers can be measured noninvasively by suitable well known non-invasive probes in the art, such as, for example, the use of a pulse oximeter for measurement of oxygen saturation. In an embodiment, invasive measurement of biochemical markers can be performed by any suitable well known invasive probes in the art, such as, for example, fluorescent probes for nitric oxide measurement and sodium and potassium probes for electrolyte measurement. In an embodiment, invasive measurement of biochemical markers can include adapting a sensory mechanism together with a delivery catheter. In an embodiment, the tissue markers can be obtained by blood testing.

### External Pressure Preconditioning

SUPERFICIAL BODY SURFACE PRECONDITIONING: As with ischemia induced by blockage of blood flow by compression over an artery such as by inflation of a blood pressure cuff, the induction of superficial pressure, to provide compression against an external body surface and thus restrict normal blood flow to the superficial tissues, can be implemented according to a schedule of transient induced pressure as required by any treatment or conditioning that may be expected. It is known that cutaneous reactive hyperemia can be produced locally to occlude the microvessels on a skin surface by applying just enough pressure to induce visible redness upon release of the pressure. Greenwood et al., “Factors Affecting the Appearance and Persistence of Visible Cutaneous Reactive Hyperemia in Man,” 1: J Clin Invest. 1948 March;27(2):187-97. Accordingly, the present inventors believe that ischemic conditioning can be provided by occluding the microvessels that are susceptible to superficial pressure and therefore empower the innate abilities of the conditioned superficial tissues for an anticipated intervention such as an incision or wound.

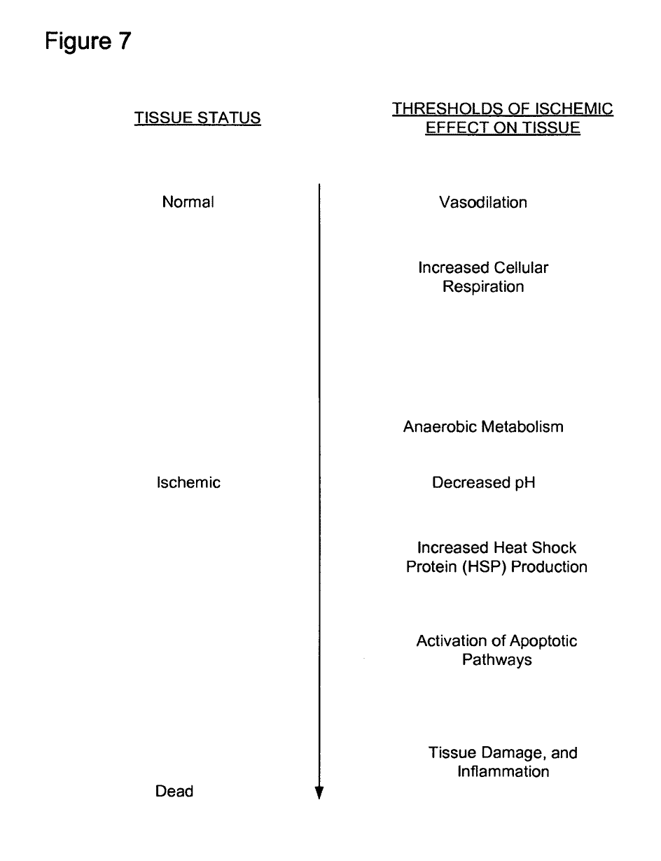

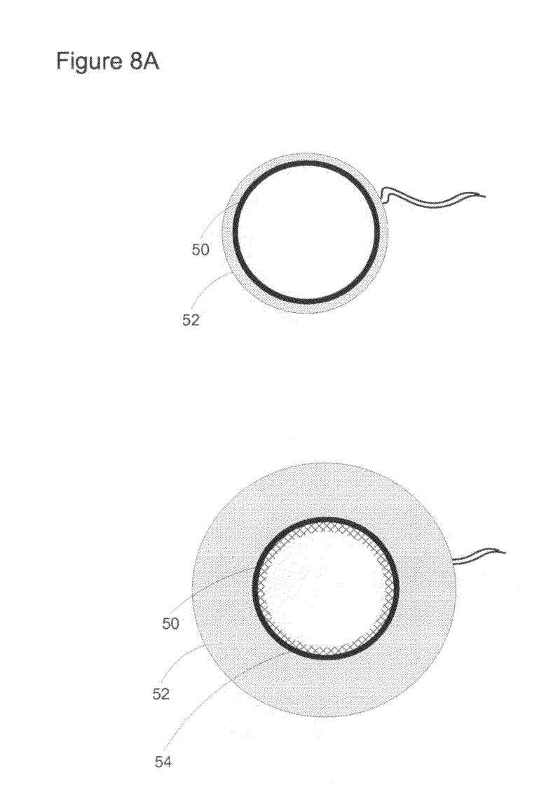

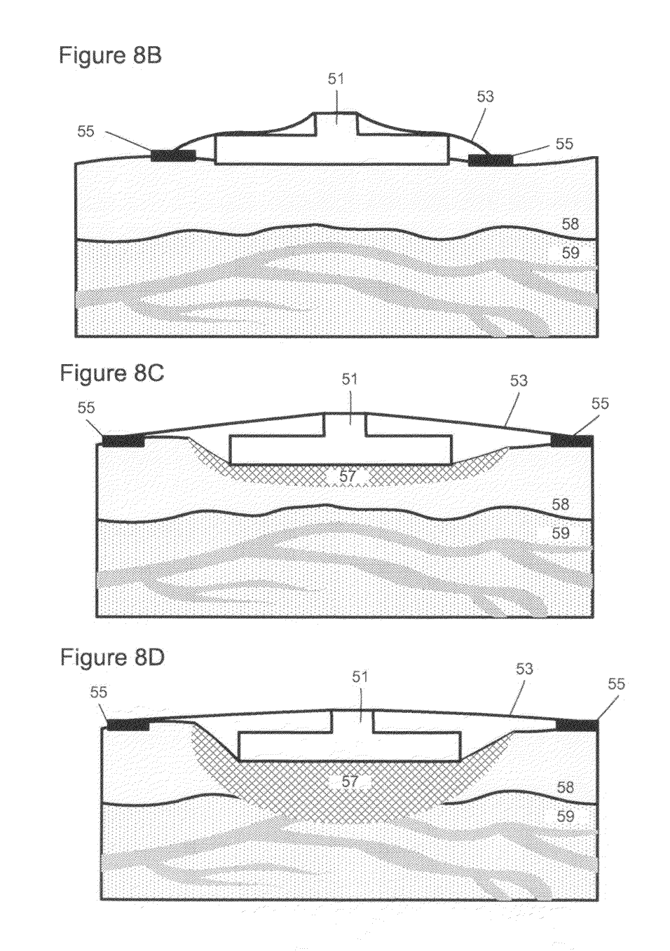

In one embodiment, the one or more administrations of superficial pressure can be provided as part of a design that includes, but is not limited to: a bed or chair, a tight-fitted garment, a pressured body suit, an adhesive wrap, an inflatable cuff, an expandable strap, or a weight, and combinations thereof. For example, FIG. 8A depicts cross sectional views of an embodiment of applying superficial pressure by an inflatable cuff (52) around an extremity (50). FIG. 8A depicts an embodiment of inflation of a cuff around an extremity to provide a small band of ischemia (54) beneath the surface of the extremity. In an embodiment, inflation of a balloon sectioned within another material such as a band that can be placed around the arm can provide localized superficial pressure around an extremity. Further, embodiments of weighted pressure and squeezing pressure can be adapted to provide pressure while being secured around an extremity.

In an embodiment, superficial pressure against a body surface such as the skin can be provided without completely wrapping around a part of the body. Such applications can be especially beneficial where proximal arterial supply is inaccessible or inconvenient, such as in applications for areas of the face, eyes, back, and chest among others. As depicted in the cross section views of FIGS. 8B-D, an occluding member (51) can be secured to a skin surface by an outer member (53) that has attaching members (55) capable of attaching to skin. As depicted, the outer member can be tightened by the attaching members to apply pressure to the occluding member. In an embodiment, the pressure applied to the occluding member can be manual, automated, combinations thereof, or any suitable in the art for the invention as described. In an embodiment, the outer member and attaching members can be part of a bandage and the occluding member can be a weight. In an embodiment, any method of applying superficial pressure can be used including but not limited to inflation, weighted pressure, and/or squeezing forces. In an embodiment, the ischemia (57) resulting from the superficial pressure can reach a dermal layer (58) alone as depicted in FIG. 8C, or also be capable of reaching subdermal layers (59) as depicted in FIG. 8D.

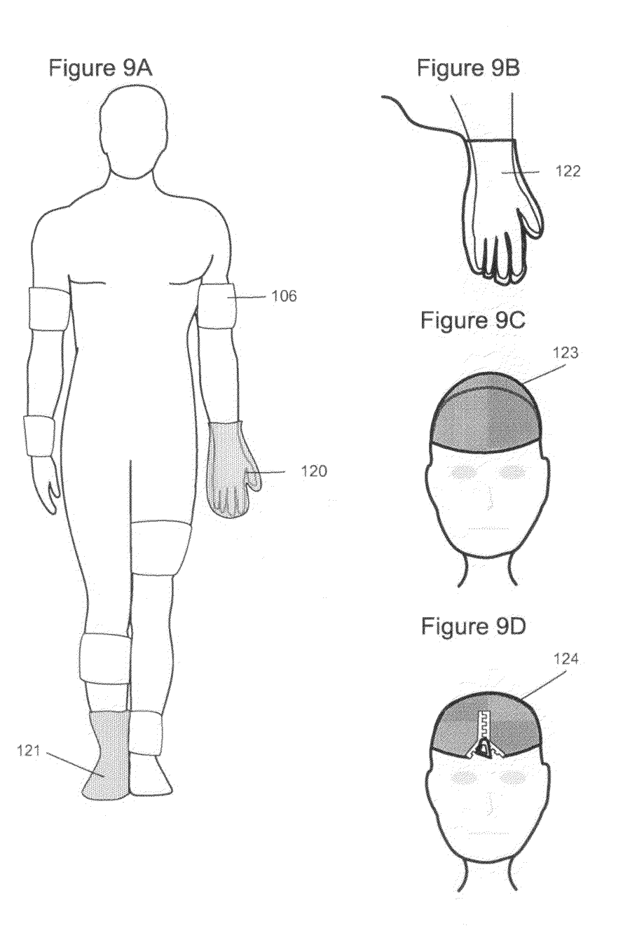

In one alternative embodiment as depicted in FIGS. 9A, 9B, 9C, and 9D, local ischemia of the superficial skin layers is provided by an inflatable mitten (120), inflatable sock (121), inflatable glove (122), inflatable cap (123), or zippered cap (124) that operates to provide compression against the skin and thus restrict normal blood flow to the superficial tissues. As with ischemia induced by blockage of blood flow by compression over an artery such as by inflation of a blood pressure cuff, the induction of superficial pressure can be implemented according to a schedule of transient induced pressure as pretreatment or preconditioning of areas that may be expected to be injured as a complication of a given medical or surgical intervention.

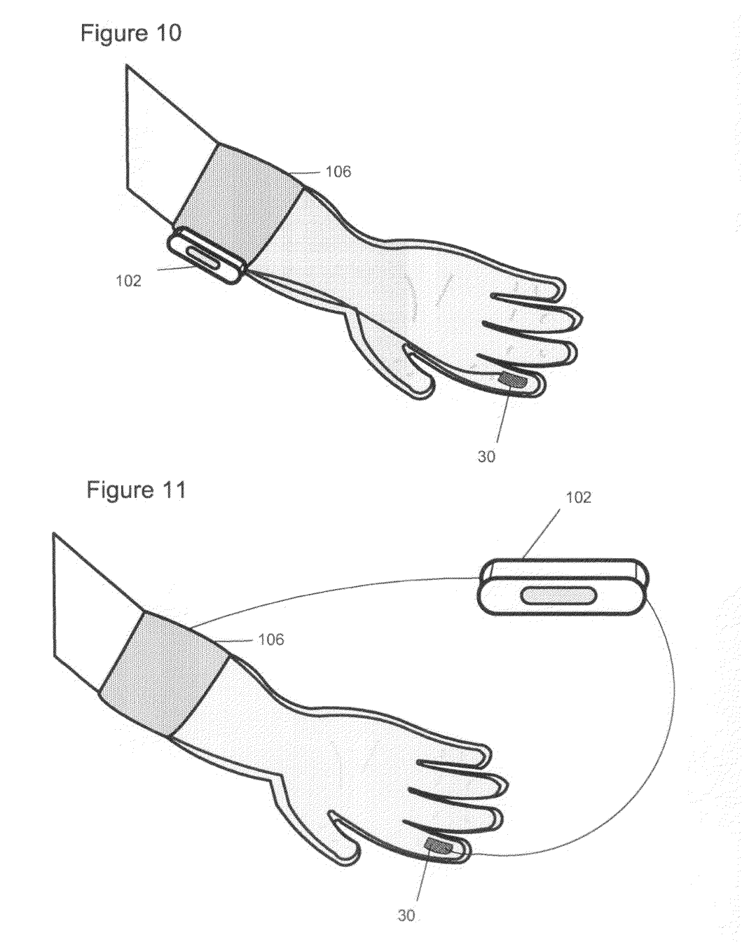

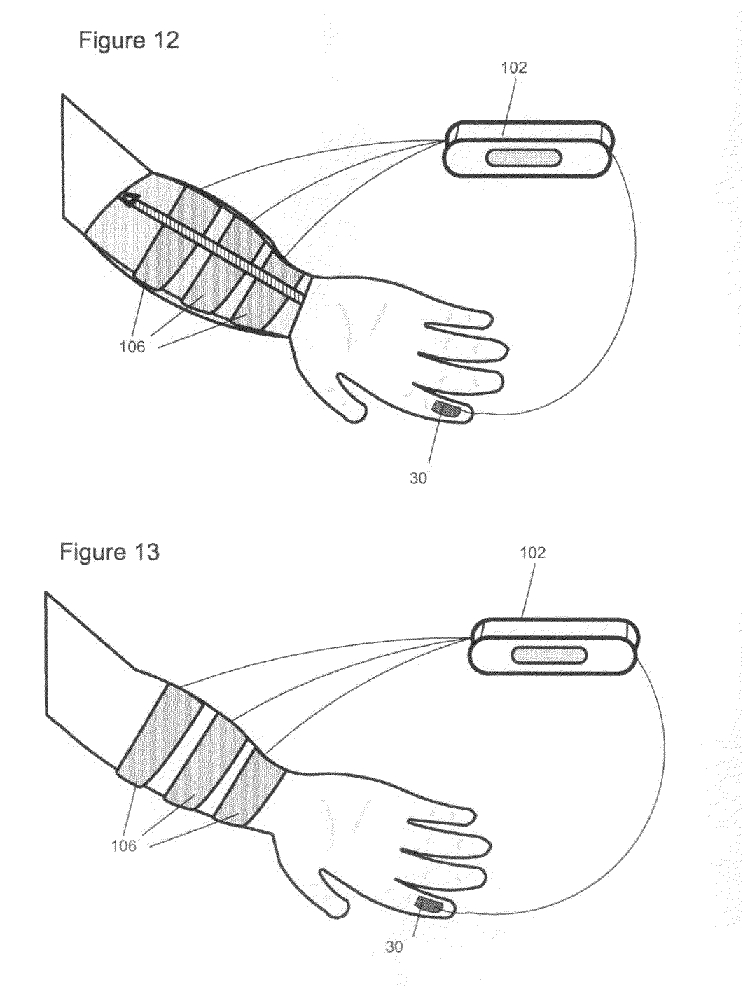

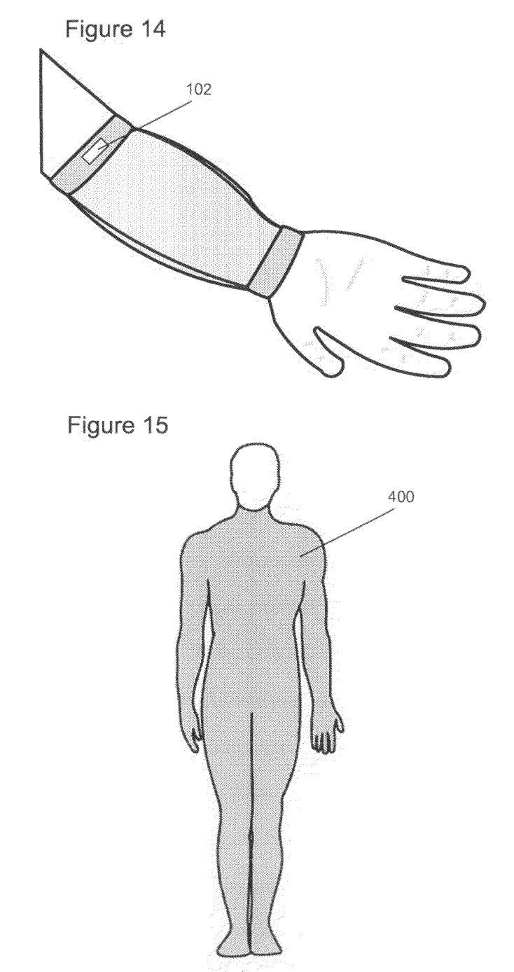

Several other embodiments for inflatable compression of the arm and hand are possible, as depicted by the illustrations of FIGS. 10-14. FIG. 10 depicts a glove adaptation with a sensor inside the glove and a controller (102) attached to the outside of the glove that controls the inflation of cuff (106). FIG. 11 depicts a glove adaptation with the sensor (30) also inside of the glove but the controller is unattached to the glove. FIG. 12 depicts a forearm glove adaptation secured to the arm with a zipper. Three cuffs (106) inside of the glove are provided to apply pressure when instructed by the unattached controller. A sensor (30) unattached to the glove is also provided for monitoring purposes. FIG. 13 depicts a forearm adaptation that is not gloved but has three cuffs and a sensor attached to a controller. FIG. 14 depicts a forearm glove adaptation that has the controller and/or monitoring integrated into a single glove device. Even further, in an embodiment, a full body suit can be used to provide ischemia to the superficial skin layers. FIG. 15 depicts an embodiment of a pressured body suit (400) that delivers external pressure to create ischemia at the skin and subdermal tissue levels.

In an embodiment, application of external superficial pressure can be provided for reduction of blood flow during the peak of blood flow during an intervention. For example, during chemotherapy, applying superficial pressure to reduce blood flow can reduce delivery of chemotherapy toxins to selected tissues. In an embodiment, applying superficial pressure to the head, e.g. via an inflatable or zippered cap, can reduce hair loss during chemotherapy by reducing the amount of toxins being delivered to hair follicles in the growth phase. In an embodiment, a cap for reducing hair loss can be adapted to fit a timer, zipper, inflation, or any other suitable apparatus to perform the invention as described herein. In an embodiment, the application of superficial pressure to reduce blood flow can be during a chemotherapy treatment. In an embodiment, applying superficial pressure during chemotherapy can be preceded by ischemic conditioning treatments before chemotherapy.

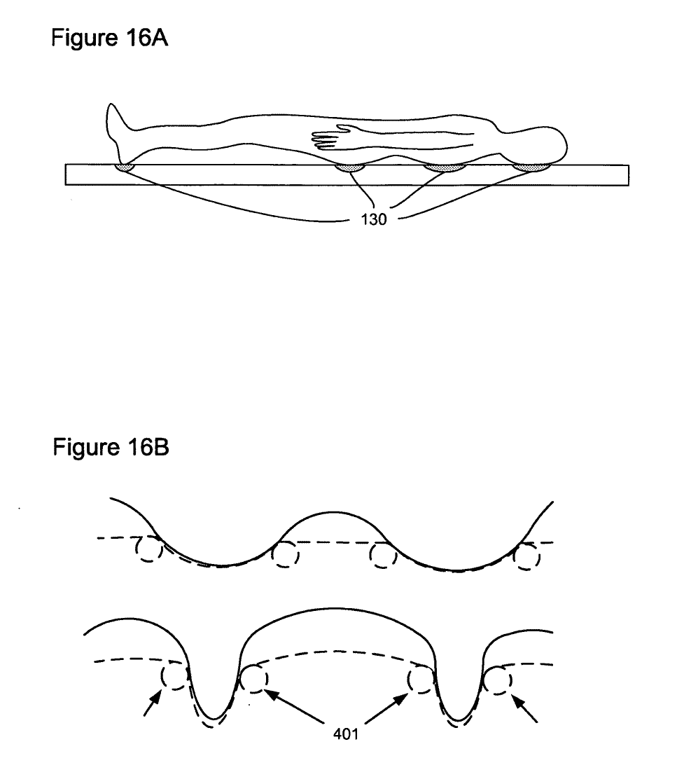

BEDSORES: In an embodiment, the invention as described herein can be particularly suited to apply superficial pressure for ischemic preconditioning of bedsores. As the skin dies, a bedsore starts as a red, painful area. Left untreated, the skin can break open and become infected. A sore can become deep, extending into the muscle, and is often very slow to heal. Pressure sores can develop on the buttocks, on the back of the head, the heels, the elbows, the hips, and/or any pressure point where the body contacts another surface. In an embodiment, a modified bed or mattress can be provided to apply superficial pressure to prevent or reduce bedsores. FIGS. 16A-B depict embodiments of a mattress capable of preventing or reducing bedsores by ischemic conditioning. In an embodiment when a patient is lying down on the mattress, the mattress can be capable of detecting pressure points (130) and treatment by an ischemic conditioning protocol using any suitable mechanism capable of applying superficial pressure, such as the skin squeezing mechanism depicted in FIG. 16B. As depicted in FIG. 16B, rollers or bars (401) are intermittently rolled together or tightened to provide transient ischemia and thus ischemic conditioning. Further, any suitable means for pressure detection or superficial pressure application that is well known in the art can be adapted for the present invention as described herein.

In an embodiment, biochemical and/or metabolic markers of tissue ischemia can be measured noninvasively by suitable well known non-invasive probes in the art, such as, for example, the use of a pulse oximeter for measurement of oxygen saturation. In an embodiment, invasive measurement of markers can be performed by any suitable well known invasive probes in the art, such as, for example, fluorescent probes for nitric oxide measurement, tissue pH probes, and sodium and potassium probes for electrolyte measurement. In an embodiment, the duration and frequency of episodes of occlusion and release can be adjusted by readings from the monitoring.

Accordingly, advantages of monitoring during occlusion include assurance of complete occlusion when needed (e.g. by monitoring pulse using Doppler probes, pulse oximeter, or other well known techniques) and also assurance of adequate levels of ischemia in the target tissue knowing the fact that different tissues experience different levels of ischemia after complete arterial occlusion.

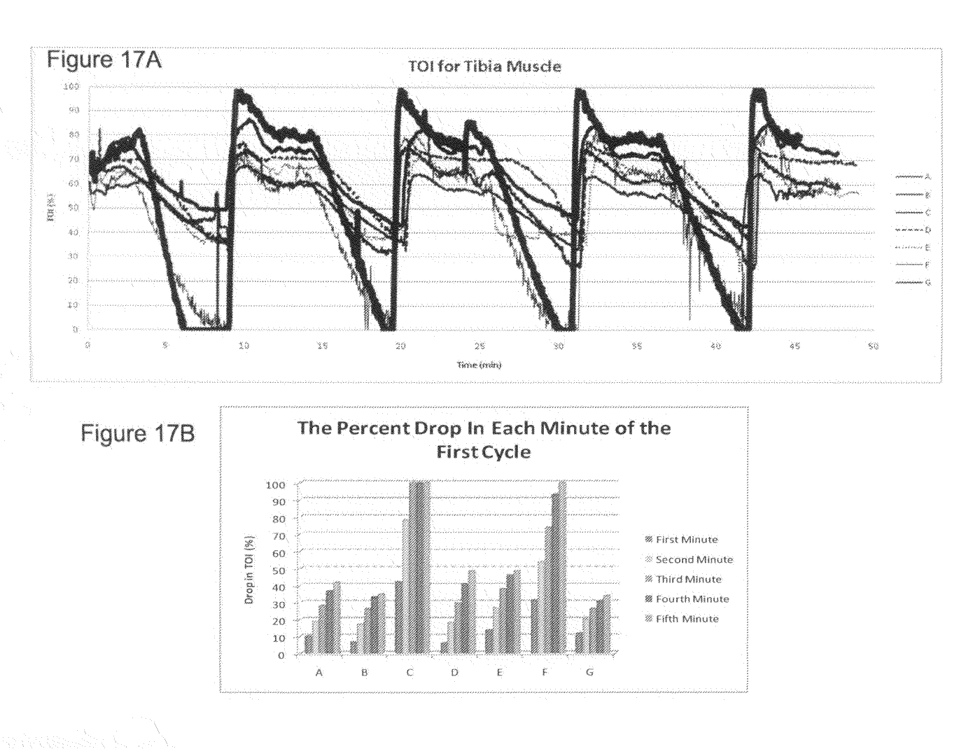

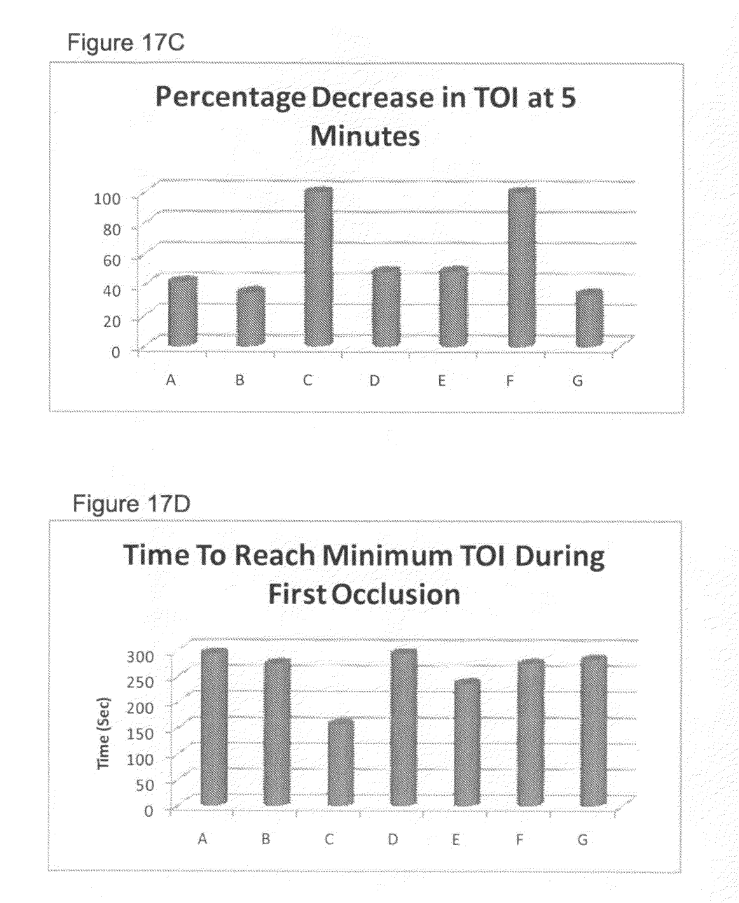

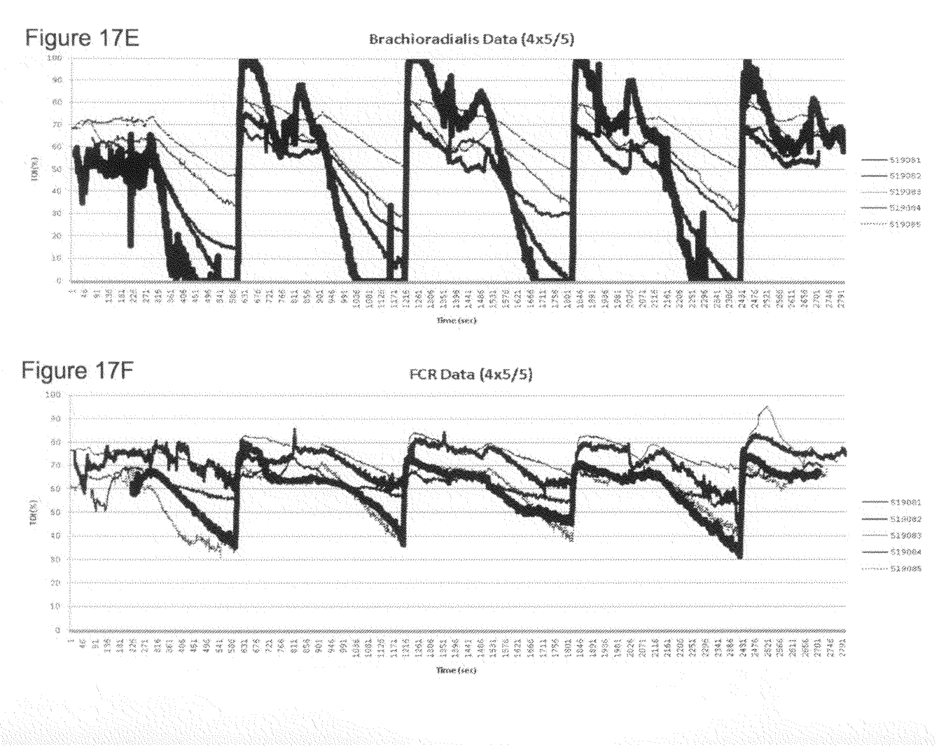

As depicted in FIGS. 17A-F, the present inventors have shown that oxygenation can vary among individuals based on a measured response of the vasculature to vascular occlusion utilizing continuous skin monitoring of oxygenation on a muscle distal (downstream) to an occluded arterial flow. A group of seven normal individuals was selected and each was subjected to four consecutive cycles of five minute occlusion followed by five minute release from a cuff placed on the mid thigh. Continuous perfusion status of the downstream tibialis anterior muscle of the lower leg was performed utilizing continuous, real-time, and direct measurement of hemoglobin oxygen saturation in tissue using near infrared (NIR) light to illuminate tissue. NIR measurement of tissue oxygenation is a well known method that analyzes the returned light and can produce a total oxygenation index (TOI), a quantitative measurement of oxygen saturation in the microcirculation of the tissue. As shown in FIG. 17A, there are significant variations in the amount of TOI during the same ischemic conditioning protocol (cycles of cuff occlusion and release) between individuals. FIG. 17B illustrates various reduction rates of TOI (the slope of drop) measured at each minute (1-5) of the first cycle during the same ischemic conditioning protocol. Further, FIGS. 17C and D clearly depict the difference between individuals in reaching minimum TOI (maximum ischemia). As depicted in FIG. 17C, the percentage drop of TOI after five minutes of cuff occlusion varies by 293.7% (100% drop versus 34% drop). Further, FIG. 17D shows the time to reach minimum TOT (maximum ischemia) ranged from 2.6 minutes to 4.9 minutes in this study group. FIG. 17E provides data obtained by the same technique but utilizing occlusion using a cuff placed over the upper arm and thus occluding the brachial artery while measuring the NIRS data over the brachioradialis muscle on several different individuals. FIG. 17F depicts data where the NIRS probe was placed over the flexi carpi radialis (FCR) muscle of the arm.

These observations clearly indicate the advantage of monitoring tissue ischemia during ischemic conditioning, so that ischemic conditioning protocols can be tailored to each individual according to their physiologic characteristics, such as metabolic rate and blood oxygenation status. Accordingly, an ischemic conditioning system benefits from monitoring of tissue ischemia by assuring the desired level of ischemia is achieved and maintained for the duration intended.

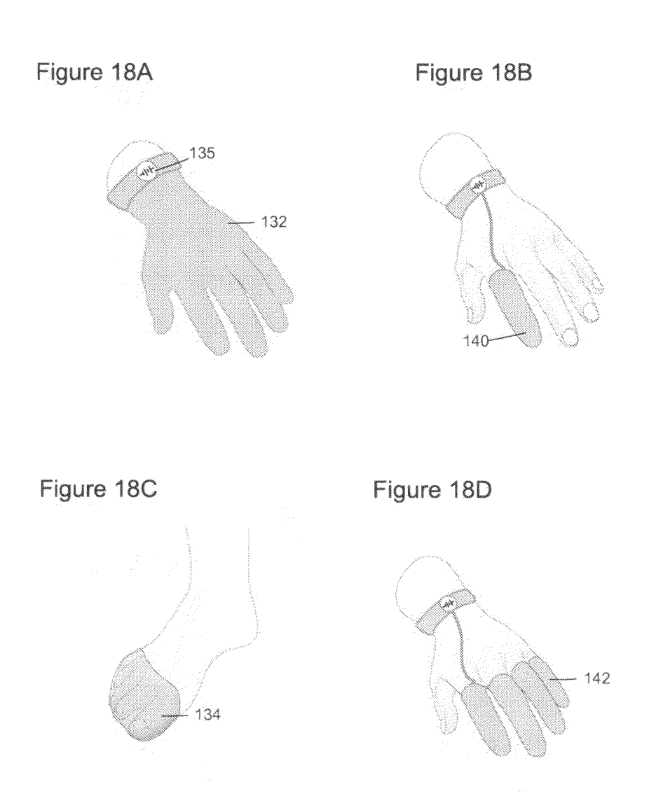

Iontophoresis: In an embodiment of the invention, conditioning is enhanced by drugs delivered to affected distal extremities by iontophoresis. As depicted in FIGS. 18A, B, C, & D, the iontophoresis delivery device can be dimensioned as a glove 132, cap 134, finger (or toe) cot 140 or plurality of cots 142. The current for driving iontophoresis can be supplied by a regulated power supply in connection with a source of line current or can be supplied by a battery such as battery 135. In an embodiment, the drug is an anesthetic drug. In other embodiments the drug is an anti-inflammatory drug. In other embodiments, the drug is an NO donor. Combinations of drugs can be selected for co-delivery depending on their shared ionic properties.

Intermittent Heating for Protection and Treatment: In an alternative embodiment, increased blood flow, enhanced metabolic activity, and anti-oxidant capability is obtained by intermittent heating of the hands and/or feet, or digits thereof. Heat is employed to shift the sympathetic-parasympathetic balance, including through the induced increase in local production of nitric oxide, in order to induce vasodilation and reduced resistance to peripheral blood flow.

In certain embodiments, the heat is provided by a wearable appliance that includes a heating element, a heating controller connected to the heating element, and a source of power for the heating element. As used herein, the term “wearable appliance” includes heatable inserts or pads that are dimensioned for placement in desired anatomical locations, including stand-alone appliances, appliances disposed in garments, and appliances that are used in association with a garment. Appliances that are used in association with a garment include appliances that are worn inside and those that are worn outside of the garment. As used herein, the term “non-wearable” appliance includes fixtures and/or portable devices that are not dimensioned to be attached or carried by an individual during ambulation. In one embodiment, local administration of heat is chronic, regular or periodic for a period prior to a planned toxic insult such as chemotherapy. For example, the individual patient may schedule a pattern of heating, such as for limited periods 5-10 times a day for a period preceding each round of chemotherapy. In another embodiment, heat is administered to the future surgical site for a period prior to orthopedic surgery. Depending on responses desired and obtained in the individual patient, the intensity and duration of heat can be tuned for optimal responses.

In one embodiment the heating method is conventional such as by electric heating coils or is provided by ultrasound, microwave (MW), radio frequency (RF) energy, and or other forms of electromagnetic energy such as infrared radiation. In other embodiments, heat is provided by a chemical reaction such as by oxidation of iron. In another embodiment, heat is provided via combustible energy sources such as butane or propane heaters. Power can be delivered through a wearable power supply and cause heat on demand.

In one embodiment ultrasound, microwave (MW) and/or radio frequency (RF) diathermy is employed to generate deep heating up to 2 inches from the skin surface without damage to the skin. The phrase “diathermy” means the controlled production of deep heating beneath the skin in the subcutaneous tissues, deep muscles and joints for therapeutic purposes. Current diathermy devices on the market generate deep heating by using radio (high) frequency, microwave or ultrasonic energy.

Ultrasound diathermy applies high-frequency acoustic vibration to tissues thereby generating heat. Current ultrasonic diathermy devices operate in a frequency range of 0.8 to 1 MH Z. MW diathermy applies a strong electrical field with comparatively low magnetic-field energy to induce intra-molecular vibration of highly polar molecules within the treated tissue to generate a thermal effect. Microwave diathermy is assigned 915 MH Z and 2450 MH Z as operating frequencies (these are also Microwave oven frequencies). RF diathermy involves application of shortwave length, high-frequency electromagnetic fields. Radio frequency (RF) diathermy is assigned an operating frequency of 27.12 MH Z (short wave) by the Federal Communications Commission. The electromagnetic field can be perpendicular or longitudinal in orientation. Although perpendicular electromagnetic field devices have been historically utilized in medical RF diathermy devices, devices able to low-energy longitudinal fields are also available (i.e. Selicor Brand Selitherm devices) and are applicable to the present invention.

The present informal position of the Food and Drug Administration is that a diathermy device should be capable of producing heat in tissue from a minimum of 104° F. to a maximum of 114° F. at a depth of two inches in not more than 20 minutes. RF heating can be done by dielectric or inductive methods and the physical configuration of the device is designed in accordance with electrical engineering principals depending on the ultrasound, MW or RF method desired.

In one embodiment of the invention, the heating is provided by Far Infrared Radiation (FIR). FIR is centered around a wavelength of 100 μm on the electromagnetic spectrum. Commercially available versions of such elements able to provide heat to subcutaneous tissue include, for example, FIR Radiant Heating elements. (Challenge Carbon Technology Co., Taiwan). Such elements are suited for FIR heated clothing due to their flat form and foldable, durable and washable properties. The elements as provided for use in clothing include lithium-ion batteries, temperature controller and OCP (Over-Charge Protector) integrated in one controller that provides for rapid heat up according to set upper levels.

In another embodiment of the invention, heat is provided by light or photo energy. In one embodiment, the photo thermal energy is provided from the red end of the visible light spectrum (greater than about 600 nm) to the near infrared end of the electromagnetic spectrum (centered about 1000 nm or 1 μm). One such suitable device and FDA approved device is available from Anodyne Therapy. The device delivers pulsed (292 times per second) near infrared (890 nm) photo thermal energy from 60 diodes mounted on flexible pads that can be placed in direct skin contact for delivery perpendicular to the surface of the skin. These devises are purported to be disclosed in U.S. Pat. Nos. Nos. 5,358,503 and 6,607,550. The photo thermal energy is applied at a level and for sufficient time to increase local microcirculation.

Counterpulsation: Alternatively or in addition to other conditioning treatments, in one embodiment counterpulsation sufficient to diminish ischemic cardiomyopathy is applied to at least one distal extremity of the patient. The counterpulsation may be performed by any suitable regimen to increase cardiac output by decreasing the afterload that the heart has to pump against and increasing the preload that fills the heart. For example, a regimen can include repetitions using series of pneumatic stockings or cuffs on legs that are connected to telemetry monitors to monitor heart rate and rhythm while the cuffs are timed to inflate at the beginning of diastole and deflate at the beginning of systole based on an electrocardiogram.

Combination Therapies: In one embodiment of the invention, at least one vascular conditioning treatment of induced ischemia or hypoxia, and/or application of heat or vibration, is combined with pharmacotherapy including by administration of a vasodilating agent, anti-oxidant, and/or anti-inflammatory agent. Multiple compounds are known in each of these categories. Existing vasodilators include for example hydralazine, ACE inhibitors (such as for example enalapril), alpha-beta blockers (such as for example carvedilol), minoxidil, and calcium channel blockers (such as for example nisoldipine, nifedipine, diltiazem and verapamil). New vasodilators such as, for example, oxdralazine are being developed and may be equally suitable. Pharmacotherapy includes agents that increase the local bioavailability of NO. The pharmacotherapy can be administered systemically or locally, such as by iontophoresis.

In another embodiment, at least one vascular conditioning treatment of induced ischemia or hypoxia, and/or application of heat or vibration, is combined with non-pharmacologic techniques for modulating the autonomic nervous system (ANS), mostly for regional and transient modulation based on anatomical reflex zones. These non-pharmacologic techniques may include non-invasive electric, magnetic, or electromagnetic neuromodulating devices used to increase local ANS activity. In another embodiment, transient intermittent ischemia and or heating is combined with hand exercises to increase demand and thereby improve nitric oxide bioavailability in the target areas.

Clinical Indications for Ischemic Conditioning: Further, several other clinical indications share the commonalities of anticipated injury, stress, inflammation, and toxicity to tissue. In an embodiment of the present invention, the inventors believe that the increase in perfusion, relaxation of smooth muscle cells, vasodilation, anti-inflammatory, and anti-oxidant effects of ischemic conditioning empowers the innate ability of tissue against anticipated insults and stressors. For example, effects of ischemic conditioning as described herein are believed to benefit treatment of inflammatory skin disorders including but not limited to eczema, dermatitis, allergic reactions, psoriasis, acne, rosacia, and/or hives. Neuropathy is also believed to be prevented or reduced by administering ischemic conditioning as described herein. For example, chemotherapy or diabetes induced neuropathy can be anticipated and is believed to be reduced or prevented by ischemic conditioning increasing the innate oxygenation and strength of nerve cells against injury. Also, ischemic conditioning as described herein of vascular tissues is believed to prevent or reduce cardiovascular and neurovascular injuries such as those associated with angina, hypertension, and transient ischemic attacks, or TIAs. Further, efficacy of immune suppressant therapies that lower the body's normal immune response are believed to be enhanced by the anti-inflammatory effects of ischemic conditioning as described herein. Accordingly, in an embodiment, enhanced treatment of pain and reduction of pain can be expected from ischemic conditioning.

Transient Ischemia for Protection Against Toxin Induced Peripheral Neuropathy: Peripheral neuropathy results from damage to the peripheral nerves, which communicate between the central nervous system (CNS) and other part of the body. All of the major nerve groups including motor, sensory and autonomic nerves can be affected with symptoms reflecting which nerve groups is affected. Peripheral neuropathy may manifest symptomatically by numbness, tingling and prickling sensations (paresthesia), burning, muscle weakness and/or loss of touch sensation in the hands and/or feet and particularly in the fingers and toes. Patients frequently experience loss of positional sense of affected body parts, weakness and leg cramping, and loss of functional utility in the hands and/or feet. Peripheral neuropathy can be either hereditary or acquired as a consequence of physical injury to a nerve, tumors, toxins, autoimmune responses, nutritional deficiencies, alcoholism, and vascular and metabolic disorders.

A significant cause of toxic neuropathy is treatment with certain chemotherapy drugs. These include the vinca alkaloids (e.g. vincristine), platinum compounds (e.g. cisplatin), Taxanes (e.g. paclitaxel), podophyllotoxins (e.g. etoposide and tenoposide), thalidomide and interferon. Chemotherapy induced peripheral neuropathy may appear suddenly or may build gradually and may worsen with each chemotherapy round. The incidence of chemotherapy induced peripheral neuropathy varies with class of chemotherapeutic agent and ranges from up to 90% with the Taxanes, 30% with thalidomide and 25% with the vinca alkaloids. Patients with chemotherapy induced peripheral neuropathy are treated symptomatically but no preventive measures are heretofore available. The high incidence of toxic sensory neuropathy coupled with lack of an effective treatment is the dose-limiting factor for these drugs.

Hypotheses for the mechanism of chemotherapy-induced neuropathy include direct toxic damage to axons and Schwann cells and disturbed cytoplasmic flow. (Quasthoff, S and Hartung, H P. “Chemotherapy-induced peripheral neuropathy” J Neurol 249 (2002) 9-17). An alternative hypothesis posits that neuropathy is a consequence of damage to the vasa nervorum or blood supply to the nerves. This hypothesis finds rational support in the fact that Taxol, thalidomide, and cisplatin exhibit anti-angiogenic properties in addition to direct effects on tumor cells. In vitro studies supporting the anti-angiogenic hypothesis have found very recent support in an in vivo study showing marked reduction of peripheral nerve intrinsic blood supply in Taxol-induced neuropathy as shown by laser-Doppler perfusion scanning and BS1 lectin staining. (R Kirchmair et al. “Therapeutic Angiogenesis Inhibits or Rescues Chemotherapy-induced Peripheral Neuropathy: Taxol- and Thalidomide-induced Injury of Vasa Nervorum is Ameliorated by VEGF” Molecular Therapy 15 (2007) 69-75). Furthermore, as reported by Kirchmair et al., intramuscular gene transfer of the endothelial mitogen VEGF-1 in proximity to the sciatic nerve prevented the expected reduction of nerve conduction velocities and of nerve blood supply when administered 2 days before Taxol injections and restored electrophysiologic nerve function. Similarly, VEGF gene therapy resulted in improvement of sensory neuropathy induced by long-term treatment of rats with thalidomide. However, as exciting these results may be, systemic angiogenic agents are contraindicated in cancer on the basis that promotion of angiogenesis might accelerate cancer growth.

In one embodiment of the present invention, intermittent transient ischemia is induced in one or more limbs of a patient preceding administration of a round of chemotherapy. The intermittent transient ischemia stimulates and conditions the downstream vasculature and thereby prevents or reduces symptoms of chemotherapy induced peripheral neuropathy.

In accordance with the novel indication of the present invention, in one embodiment the body's own adaptive responses to induced ischemia or hypoxia are harnessed to provide protection against peripheral neuropathy induced by chemotherapy as well as other injurious mechanisms. In accordance with the method disclosed herein, a scheduled series of transient ischemic episodes is administered prior to administration of chemotherapy, including if desired, between rounds of chemotherapy. The transient ischemic episodes provide protection against the incidence and extent of peripheral neuropathy by several mechanisms including without limitation: increased scavenging of free radicals induced by the chemotherapy, reduction in inflammation, and increased functional capillary density, particularly in the hands and feet. In another embodiment of the invention, a scheduled series of transient ischemic episodes is applied to treat existing peripheral neuropathy. Further, in an embodiment, ischemic conditioning can similarly protect against other anticipated stressors, including but not limited to endotoxins, such as LPS, responding to stress and/or infection.

In one embodiment of the invention, transient ischemia is implemented by cuffs or straps (106) that are secured around one or more of the limbs of the patient, as depicted in FIG. 3C. As shown in FIG. 3C, the occlusive cuffs or straps can be placed over one or more locations for compression sufficient to occlude blood flow to the hands and or feet. For example, for occlusion of blood supply to the hands, compression can be applied to the upper arm over the brachial artery, the lower arm over the radial and ulnar arteries, or the wrist over the radial artery. For occlusion of bloods supply to the feet, compression can be applied to the upper leg over the femoral and deep femoral arteries, or to the lower leg over the tibial arteries. In one embodiment, the cuffs are inflatable and inflation results in sufficient pressure around the circumference of the limb to result in occlusion of the arterial blood supply to the limb.

The duration of ischemia varies by therapeutic targets, but is typically provided for a period from about 1 to about 20 minutes, preferably from about 2 to about 5 minutes, followed by release of the occlusion. Occlusion and release (reactive hyperemia) procedures with different occlusion times are implemented depending on individual tolerance and response to therapy as well as the planned chemotherapy schedule such that a desired distal and or contralateral vascular/neuro/neurovascular function is obtained. Repeated cuff occlusion and release is tailored to improve vasoreactivity (increasing the vasodilative capacity) by improving nitric oxide bioavailability (reducing destruction or increasing production). This effect can be seen in the same distal extremity as the cuff inflation but is also expected to have neurovascular mediated vasodilation of the contralateral extremity as well.

Ischemic Conditioning to Reduce Perioperative Complications: Consideration of perioperative complications is critical before a surgical procedure. For example, cardiovascular disease and pulmonary disease are both associated with poor outcome of surgery. Intraoperative complications during surgery, e.g. hemorrhage or perforation of organs, can have lethal sequelae. Numerous postoperative complications also exist. For example, local infection of the operative field is possible. Acute respiratory distress syndrome (ARDS) and hypostatic pneumonia due to shallow inspirations frequently occur especially in patients recovering from abdominal surgery, including but not limited to valvular surgery, lung resection, esophagus resection, and/or vascular surgery. Cerebrovascular accidents also occur at a higher rate during the postoperative period.

The Complex Regional Pain Syndromes (CRPS type I, formerly Reflex Sympathetic Dystrophy or RSD, and CRPS type II, formerly Causalgia) define region specific, chronic and often incapacitating pain that develops following a noxious insult. Periods of immobilization or invasive procedures such as surgery or venipuncture have been identified as precipitating CRPS. CPRS can be a complication of orthopedic surgery with estimates of incidence of 2.3-4% after arthroscopic knee surgery, 2.1-5% after carpal tunnel surgery, 13.6% after ankle surgery, 0.8-13% after total knee arthroplasty, 7-37% for wrist fractures and 4.5-40% after fasiectomy for Dupuytren contracture. In addition, as many as 6-10% of patients that have CRPS will require surgery or further surgery and recurrence is common. D C Warltier, “Preventing the Development of Complex Regional Pain Syndrome after Surgery” Anesthesiology 101 (2004) 1215-24. Regardless of the suspected cause, the extent of pain, inflammation and subsequent nerve damage is abnormal and highly exaggerated relative to the precipitating event. In some cases, CRPS results in denervation and may necessitate amputation. Current therapies include physical therapy combined with administration of anti-epileptics, opioids, antidepressants, implantable devices or sympathetic nerve blockade. The uncertain etiology of the disease has heretofor suggested no preventive strategies.

Surgery induced CRPS is not predictable yet occurs following a significant number of orthopedic surgical procedures. However, because the condition cannot be predicted and doesn't affect a majority of surgical patients, any potentially dangerous or expensive preventive therapy would not be indicated. The present method of administering one or more transient ischemic episodes to the limb that will be treated by surgery is neither dangerous nor expensive and may be readily implemented as a preventative measure in every patient. The transient ischemic episodes provide protection against the incidence and extent of peripheral nerve damage by several mechanisms including without limitation: increased scavenging of free radicals induced by the trauma of surgery and reduction in inflammation. If administered in a series of episodes sufficiently in advance of surgery, the method is expected to increase functional capillary density in desired surgical site and may be further expected to hasten wound healing. In another embodiment of the invention, a scheduled series of transient ischemic episodes is applied to treat existing CRPS.

In an embodiment, the present invention as described herein aims to strengthen tissues under perioperative conditions. In one embodiment of the present invention, intermittent transient ischemia is induced in one or more limbs of a patient. The intermittent transient ischemia stimulates and conditions the vasculature and thereby prevents or reduces perioperative complications.

For example, kidney damage can be reduced and/or prevented by ischemic conditioning. In an embodiment, ischemic conditioning can reduce or prevent acute kidney injury during and after major surgeries such as cardiac bypass, vascular surgeries, and aortic aneurysm surgery. In an embodiment, contrast-dye induced nephrotoxicity can be reduced and/or prevented upon ischemic conditioning of a patient prior to injection of a damaging contrast dye for angiographic procedures including but not limited to percutaneous coronary intervention (PCI) and diagnostic coronary angiography.

Thus, in an embodiment, multiple separate ischemic conditioning treatments can be scheduled in any suitable manner prior to, during, and/or after surgery as described herein, including but not limited to: several times daily, frequently over extended periods of time, based on assessments of specific interventions and/or treatment resistance, and combinations thereof. Further, in an embodiment, one or more of the ischemic conditioning treatments can be administered remotely from the operative tissue and provide a systemic effect. For example, occlusive cuffs can perform ischemic conditioning on an extremity, such as an arm or leg, to improve postoperative healing from an incision in a part of the body that is difficult to access for occlusion, like the back, chest, or torso.

Ischemic Conditioning to Improve Wound Healing: Wound healing is an important health care problem. Determining whether a wound is acute or chronic is the first step in understanding the components of healing or lack of healing. Medical wounds can vary from being acute to chronic, or occurring following a repeated or persistent pattern. The acute care wound model of healing includes hemostasis, inflammation, proliferation, maturation, and is unique from chronic wound management. Chronic wounds are wounds that have failed to proceed through an orderly and timely process to produce an anatomic and functional integrity, or proceed through the repair process without establishing a sustained and functional result.

However, because each condition cannot be predicted and has variations for different patients, any ischemic conditioning therapy can be modified to suit the unique parameters for any particular condition. The present method of administering one or more transient ischemic episodes to the limb according to a schedule is neither dangerous nor expensive and may be readily implemented in every patient. The transient ischemic episodes provide protection and treatment against medical wounds by several mechanisms including without limitation: increased nitric oxide bioavailability, increased scavenging of free radicals and reduction in inflammation. If administered in a series of episodes over a sufficiently amount of time, the method is expected to increase arterial and smooth muscle flexibility, functional capillary density, and to hasten wound healing.

In an embodiment of the invention, the duration and frequency of ischemia targeted toward a tissue that is wounded or to be wounded may have a relationship with the effect of wound healing. Similar to perioperative outcomes, desired therapeutic effects within an early window and a delayed window of protection after conditioning are expected. Thus, in an embodiment, multiple separate ischemic conditioning treatments can be scheduled in any suitable manner as described herein, including but not limited to: several times daily, frequently over extended periods of time, based on assessments of specific interventions and/or treatment resistance, and combinations thereof. Further, in an embodiment, one or more of the ischemic conditionings directed towards acute wounds can be administered remotely from the targeted tissue that is wounded or to be wounded and provide a systemic effect. For example, occlusive cuffs can perform ischemic conditioning on an extremity, such as an arm or leg, to improve wound healing from an anticipated incision in a part of the body that is difficult to access for occlusion, like the back, chest, or torso.

In an embodiment of the invention, a scheduled series of transient ischemic episodes can be applied as conditioning to prevent or manage chronic wounds. Of the numerous compounds that are released following an ischemic episode as described herein, several may improve response to any wound or injury. For example, an increase in nitric oxide and adenosine bioavailability is known to occur after an ischemic episode. These compounds are frequently targeted by drug therapies and are well known to relax smooth muscle cells, decrease arterial stiffness, and improve wound healing over time. Accordingly, ischemic conditioning is able to noninvasively simulate ischemic effects of existing therapies. In an embodiment, ischemic conditioning can be administered supplemental to, or in addition to, conventional treatments of chronic wounds, such as heating, drugs, and irrigation.

For chronic wound treatment, separate ischemic conditioning treatments can also be scheduled in any suitable manner as described herein, including but not limited to: several times daily, frequently over extended periods of time, based on assessments of specific interventions and/or treatment resistance, and combinations thereof. Further, in an embodiment, one or more of the ischemic conditionings directed towards chronic wounds can be administered remotely from the targeted tissue that is wounded or to be wounded and provide a systemic effect. For example, occlusive cuffs can perform ischemic conditioning on an extremity, such as an arm or leg, to improve wound healing from an anticipated chronic wound.

In an embodiment, several tissue injuries resulting from chronic wounds can benefit from scheduled ischemic conditioning and the resulting increase in perfusion, relaxation of smooth muscle cells, vasodilation, anti-inflammatories, and anti-oxidants. For example, the vast majority of chronic wounds can be classified into three categories: venous ulcers, diabetic, and pressure ulcers. Venous ulcers, which usually occur in the legs, are thought to be due to venous hypertension caused by improper function of valves that exist in the veins to prevent blood from flowing backward. Ischemia often results from the dysfunction and, combined with reperfusion injury, causes the tissue damage that leads to the wounds.

Another major cause of chronic wounds, diabetes, is increasing in prevalence. Diabetics have a higher risk for amputation than the general population due to chronic ulcers. Diabetes also causes neuropathy, which inhibits the perception of pain. Thus patients may not initially notice small wounds to legs and feet, and may therefore fail to prevent infection or repeated injury, such as in the case for diabetic foot injuries. Further, diabetes causes immune compromise and damage to small blood vessels, preventing adequate oxygenation of tissue, which can cause chronic wounds. Pressure also plays a role in the formation of diabetic ulcers.

Other leading types of chronic wounds are pressure ulcers, which usually occur in people with conditions such as paralysis that inhibit movement of body parts that are commonly subjected to pressure such at the heels, shoulder blades, and sacrum. Pressure ulcers are caused by ischemia that occurs when pressure on the tissue is greater than the pressure in capillaries, and thus restricts blood flow into the area. For example, a bedsore develops when an area of the skin is under pressure and the blood supply to the skin is cut off for more than a few hours. Further, muscle tissue, which needs more oxygen and nutrients than skin does, shows some of the worst effects from prolonged pressure. Reperfusion injury damages tissue in pressure ulcers as in other chronic wounds.

Further, microvascular dilative capacity is hindered and inflammation is increased in Raynaud's syndrome and several associated disorders such as scleroderma (a collagen-related immune disorder) and small vessel vasculitis (disorders of anti-neutrophil cytoplasmic antibodies, or ANCAs, for example). Further, diabetes, insulin resistance, high blood glucose, and several other metabolic disregulations are well known to exacerbate inflammation and oxidation. In an embodiment, ischemic conditioning can allow for detection and management of subclinical diabetic neuropathy. Comparing nerve conduction differences before and after an episode of ischemic conditioning can provide information about existence and amounts of nerve damage due to diabetic microvascular injury. Any suitable nerve function measurement, such as needle electromyography (EMG), can be used.

Tissue Conditioning for Transplants, Implants, and Grafting: In an embodiment, the invention as described herein can be particularly suited to apply ischemic conditioning to reduce complications and/or improve outcomes for organ or tissue transplants, implants, and/or grafts. A transplant is the moving of a whole or partial organ from one body to another or from a donor site on the patient's own body, for the purpose of replacing the recipient's damaged or failing organ with a working one from a donor site. Donor tissue can be living or deceased. Generally, transplants can be categorized into organ transplants and tissue transplants. Examples of organs that can be transplanted are the heart, kidneys, liver, lungs, pancreas, and intestine. Examples of tissues include bones, tendons, cornea, heart valves, veins, and skin. Further, in medicine, grafting is a sensitive surgical procedure to transplant tissue without a blood supply. The implanted tissue must obtain a blood supply from the new vascular bed or otherwise die. The term is most commonly applied to skin grafting, however many tissues can be grafted, including but not limited to: skin, bone, nerves, tendons, and cornea.

Animal research has shown that ischemic preconditioning protects grafts from subsequent long-term cold preservation-reperfusion injury. See e.g. Yin et al., “Protective effect of ischemic preconditioning on liver preservation-reperfusion injury in rats,” Transplantation. 1998 Jul. 27;66(2):152-7 [a rat liver transplantation model]. Further, in a recent 2007 publication, remote ischemic preconditioning has been shown to clinically benefit patients undergoing coronary artery bypass graft (CABG) interventions. Hausenloy et al., “Effect of remote ischaemic preconditioning on myocardial injury in patients undergoing coronary artery bypass graft surgery: a randomised controlled trial,” Lancet 2007; 370: 575-79.

However, the present inventors believe ischemic conditioning protocols can be improved for transplants, implants, and/or grafts. For example, ischemic conditioning of donor cells prior to the intervention is believed to strengthen tissue and improve their survival after transplantation. In an embodiment, donor tissue therapies such as hypothermia and/or preservatives can be enhanced by ischemic conditioning of that donor tissue. In an embodiment, cardioplegia procedures in particular can benefit from ischemic conditioning and strengthening of the cardiac tissue which will have activity temporarily ceased. In an embodiment, duration and frequency of multiple administrations of ischemic conditioning can be optimized for a planned intervention. In an embodiment, direct and/or remote ischemic conditioning of donor cells can be provided prior to a transplant, implant, or graft. Further, ischemic conditioning of the recipient tissue can also be provided. Considering skin grafts in particular, in an embodiment, the donor and/or host tissue of a skin graft or skin flap can undergo superficially pressured ischemic conditioning according to an optimized protocol to improve outcomes of grafting.

1. A method for an ischemic conditioning treatment in a patient comprising:
causing ischemia by interrupting blood flow to a tissue,
sensing markers of one or more of ischemia, blood flow, and metabolism in the tissue, and
adjusting the ischemia based on the sensed results.

2. The method of claim 1, wherein the sensed markers of ischemia include one or more of tissue oxygenation, and levels of hemoglobin, and the sensed markers of metabolism include one ore more of lactate, pH, oxygen, carbon dioxide, ATP, ADP, adenosine, cytochrome oxidase, redox voltage, erythropoietin, bradykinin, and opioids.

3. The method of claim 1, wherein the adjusting of ischemia is a change of an extent, duration, frequency, or combinations thereof, of ischemia in each ischemic conditioning treatment.

4. The method of claim 1, further comprising monitoring of markers of response to the ischemic conditioning treatments.

5. The method of claim 4, wherein the monitoring of markers of response to the ischemic conditioning treatments changes or stops the extent, duration, frequency, or combinations thereof, of ischemic conditioning treatments.

6. The method of claim 1, wherein the monitoring of markers of ischemia is obtained from non-ischemic tissue

7. The method of claim 6, wherein the monitoring of markers of ischemia from non-ischemic tissue is monitoring of circulating blood lactate.

8. The method of claim 1, wherein multiple ischemic conditioning treatments are stacked to provide an increase in early and delayed SWOP protective effects.

9. The method of claim 1, wherein the monitoring is a measurement of pulse, blood flow, or combinations thereof.

10. The method of claim 1 wherein the tissue is the anticipated location of a tissue injury, including one or more of: toxin induced peripheral neuropathy, a site of a surgical procedure, an anticipated pressure induced sores, a site of toxic shock, and a site of infection.

11. The method of claim 1 wherein the tissue is a donor or recipient tissue for a transplant, implant, graft, or combination thereof.

12. The method of claim 1 wherein the ischemic conditioning treatment is performed preoperatively, intraoperatively, postoperatively, and/or combinations thereof.

13. The method of claim 1 wherein the ischemic conditioning treatment is augmented with a supplemental therapy selected from the group consisting of: heat, photo thermal energy, hypoxia, hypothermia, pressure, vital sign measures, blood pressure measures, vibration, pain management, treatment drugs including inhalational anesthetics, anti-inflammatory agents, compounds that increase the bioavailability of nitric oxide (NO), and combinations thereof.

14. A device for inducing ischemic conditioning, comprising:
an occluding member adapted to occlude a blood vessel and cause ischemia;
a sensor to monitor a response to the ischemic conditioning treatments, wherein the monitored response is one or more of markers of ischemia, blood flow and metabolism in a tissue distal to the occlusion;
a programmable controlling member to control frequency and duration of ischemia in a tissue according to an ischemic conditioning protocol based on the monitored response; and
a data storage member to store the ischemic conditioning protocol, monitored response, and/or combinations thereof.

15. The device of claim 14 wherein the sensor is capable of monitoring markers of ischemia such as tissue oxygenation; markers of metabolism including lactate, pH, oxygen, carbon dioxide, ATP, ADP, adenosine, cytochrome oxidase, redox voltage, erythropoietin, bradykinin, opioids; and markers of blood flow or pulse; or combinations thereof.

16. The device of claim 14 wherein the programmable controlling member is programmed with an ischemic conditioning protocol based on a reference algorithm.

17. The device of claim 16 wherein the algorithm results from a multivariate prediction model of the extent of tissue ischemia during vessel occlusion based on one or more variables including variables selected from the group consisting of: age, gender, body mass index, body fat percentage, basal metabolic rate, heart rate, limb circumference, and combinations thereof.

18. A device for inducing ischemic conditioning, comprising:
an occluding member adapted to occlude a blood vessel and cause ischemia;
a programmable controlling member to control frequency and duration of ischemia in a tissue according to a protocol based on an algorithm that is obtained from a multivariate prediction model of the extent of tissue ischemia during vessel occlusion; and
a data storage member to store the ischemic conditioning protocol, monitoring results, or combinations thereof.

19. The device of claim 18, wherein the algorithm is based on one or more variables comprising variables selected from the group consisting of: age, gender, body mass index, body fat percentage, basal metabolic rate, heart rate, limb circumference, and combinations thereof.

20. The device of claim any one of claims 18 and 19, wherein the occluding member is an inflatable cuff.

21. The device of claim 18, wherein the ischemic conditioning protocol in the programmable controlling member is programmed by a separate device.

22. The device of any one of claims claim 18-21, further comprising a display to show the ischemic conditioning protocol, stored data, results of the ischemic conditioning, or combinations thereof.

23. The device of any one of claims 18-22, further comprising one or more additional measurement devices capable of measuring vital signs and/or blood pressure.

24. A method for an ischemic conditioning treatment in a patient comprising:
causing ischemia by interrupting blood flow to a tissue utilizing an controlled occluding device, wherein the blood flow is interrupted at least once in a period of 72 hours prior to an anticipated injury, surgical intervention, or medical intervention,
monitoring one or more of tissue markers of ischemia; markers of metabolism; and markers of blood flow or pulse in the patient, and
adjusting the extent, duration, frequency, or combinations thereof, of ischemia based on the monitoring results.

25. The method of claim 24, further comprising interrupting blood flow to a tissue at least once in a period of 72 hours after an injury, surgical intervention, or medical intervention.

26. The method of claim 24 wherein the sensed markers of ischemia include one or more of tissue oxygenation, and levels of hemoglobin, and the sensed markers of metabolism include one ore more of lactate, pH, oxygen, carbon dioxide, ATP, ADP, adenosine, cytochrome oxidase, redox voltage, erythropoietin, bradykinin, and opioids.
